(19) United States
(12) Patent Application Publication
(10) Pub. No.: US 2025/0283173 A1
(43) Pub. Date: Sep. 11, 2025

(54) GENE FUSIONS ASSOCIATED WITH AMYOTROPHIC LATERAL SCLEROSIS (ALS)

(71) Applicant: The General Hospital Corporation, Boston, MA (US)

(72) Inventors: Ghazaleh Sadri-Vakili, Watertown, MA (US); Merit E. Cudkowicz, Newton, MA (US); James D. Berry, Belmont, MA (US); Tiziana Petrozziello, Medford, MA (US); Khashayar Vakili, Watertown, MA (US); Ernest Fraenkel, Newton, MA (US); Yogindra Raghav, Breinigsville, PA (US); Allison Dilliott, Montreal (CA); Sali Farhan, Montreal (CA); Michael E. Talkowski, Lexington, MA (US); Dadi Gao, Cambridge, MA (US); Ricardo Mouro Pinto, Boston, MA (US)

(21) Appl. No.: 18/861,563

(22) PCT Filed: May 1, 2023
(86) PCT No.: PCT/US2023/066450

§ 371 Date: Oct. 29, 2024

Related U.S. Application Data
(60) Provisional application No. 63/337,025, filed on Apr. 29, 2022.

(51) Int. Cl. C12Q 1/6883; C12Q 1/6869

(52) U.S. Cl. CPC C12Q 1/6883; C12Q 1/6869; C12Q 2600/156; G01N 2800/50

(57) ABSTRACT

Provided herein are methods for diagnosing ALS, or determining risk of developing ALS. The methods include detection of a fusion as described herein. The methods can include detecting genomic fusions, fused transcripts, or fusion proteins (where proteins are produced) as described herein.

### CLAIM OF PRIORITY

This application claims the benefit of U.S. Provisional Application Ser. No. 63/337,025, filed on Apr. 29, 2022. The entire contents of the foregoing are incorporated herein by reference.

### TECHNICAL FIELD

Provided herein are methods for diagnosing ALS, or determining risk of developing ALS. The methods include detection of a gene fusion described herein. The methods can include detecting genomic fusions, fused transcripts, or fusion proteins (where proteins are produced) as described herein.

### BACKGROUND

Amyotrophic lateral sclerosis (ALS) is a lethal, adult-onset, neurodegenerative disease primarily affecting motor neurons in the motor cortex, brainstem, and spinal cord.^{1,2} Genetics is an important risk factor for ALS, as 40-55% of familial ALS (fALS) are due to known genetic mutations,³ and over 50 causative or disease-modifying genes have been identified that are linked to disease, including but not limited to superoxide dismutase 1 (SOD1), TAR DNA binding protein (TARDBP), fused in sarcoma (FUS), and a hexanucleotide repeat expansion in C9orf72.4,5 In addition, genetic risk factors also contribute to sporadic ALS (sALS); however, the causes of more than 80% of cases remains unknown.⁴

### SUMMARY

Provided herein are methods that can include providing a sample, preferably a sample comprising genomic DNA (gDNA) and/or RNA, optionally non-coding mRNA and/or mRNA, from a human subject, and detecting presence of a fusion described in any of Tables 2-7 in the gDNA and/or RNA. Also provided herein are methods for diagnosing amyotrophic lateral sclerosis (ALS) or risk of developing ALS in a subject; the methods comprise detecting presence of a fusion described in any of Tables 2-7. In some embodiments, only one of gDNA or RNA is assayed for the presence of a fusion; in others, both gDNA and RNA are assayed for the presence of a fusion.

In some embodiments, the fusion is a fusion of YAF2 and RYBP genes.

In some embodiments, the subject is suspected of having or at risk of having amyotrophic lateral sclerosis (ALS).

In some embodiments, the sample comprises serum or cerebrospinal fluid, or comprises gDNA or RNA isolated from whole blood, plasma, serum or cerebrospinal fluid.

In some embodiments, the methods further comprise isolating exosomes comprising proteins or nucleic acids from the sample, and detecting the presence of a fusion in the exosomes.

In some embodiments, detecting presence of a fusion comprises using RNA-seq and/or long-read sequencing to detect RNA transcripts of a fusion.

In some embodiments, detecting presence of a fusion comprises contact the sample, e.g., contacting the gDNA and/or RNA in the sample or isolated from the sample, with a plurality of oligonucleotides that bind to a fusion sequence. In some embodiments, the fusion comprises a fusion of two or more genes, and the plurality comprises oligonucleotides that bind to each of the two or more genes.

In some embodiments, the fusion is a fusion of YAF2 and RYBP genes, and the plurality comprises oligonucleotides that bind to YAF2 and oligonucleotides that bind to RYBP.

In some embodiments, each of the plurality of oligonucleotides comprise a primer for sequencing, and the method comprises determining a sequence of a portion of the gDNA and/or RNA.

In some embodiments, each of the plurality of oligonucleotides is suitable for priming amplification, and the method further comprises amplifying a portion of the gDNA and/or RNA using oligonucleotides bound to the gDNA and/or RNA as primers.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. Circos plot of breakpoint-unique gene fusions identified in ALS and control samples using RNA-Seq datasets. Representation of significant fusion events where the width on the end of each line segment indicates the portion of the chromosome involved in the fusion event. Chromosomes were expanded 200× for clearer visualization and gene fusions on each are shown. Gene fusions are represented by lines on each chromosome that were expanded 10× for clearer visualization and include: ALS unique fusions, local rearrangements, not close-proximity, and all others.

FIGS. 2A-D. Characterization of the breakpoint-unique gene fusions and their subtypes in ALS and control samples (n=1542 and n=249, respectively). The breakpoint-unique gene fusions in ALS and control samples were compared to determine the distribution of (A) intra-chromosomal and inter-chromosomal gene fusions, (B) intra-chromosomal gene fusion subtypes, (C) fusion events per sample based on the chromosome(s) involved, and (D) the proportion of fusion events per chromosome(s) involved in the gene fusions corrected for total number of genes located on the chromosome (Ensembl, release 106).

FIGS. 3A-B. Breakpoint-unique gene fusions carried in ALS and control samples (n=1542 and n=249, respectively). The distribution of breakpoint-unique gene fusions carried per sample was compared between ALS and control samples using the Welch's t-test, both independent of sample tissue source and within each individual tissue source. (A) ALS samples carried significantly more breakpoint-unique gene fusions (mean=14.18; SD=6.54) than controls (mean=11.16; SD=6.10) (p=4.505e-12). (B) Significantly more breakpoint-unique gene fusions were carried by ALS samples compared to controls in the cervical spinal cord (p=0.0022), lumbar spinal cord (p=0.0012), frontal cortex (p=0.0011), temporal cortex (p=2.375e-4), hippocampus (p=0.0056), and cerebellum (p=1.393e-4). No statistical comparisons were performed in the thoracic spinal cord, sensory cortex, or occipital cortex as there were too few (n<10) tissue samples from controls. *<0.05; **<0.01; *** p<0.001.

FIGS. 4A-B. Enrichment of breakpoint-unique gene fusions carried by ALS and control samples. Welch's t-test was used to compare the number of breakpoint-unique gene fusions of each subtype carried by each ALS and control sample both independent of sample tissue source and within each individual tissue source. (A) There was a significant enrichment of all four intra chromosomal subtypes in ALS compared to controls, including local rearrangements (p=8.979e-15), not close proximity fusions (p=0.0105), neighbor fusions (p=2.930e-09), and overlapping neighbor fusions (p=0.0151). (B) Following subgrouping of gene fusion based on the tissue source of the sample in which they were identified, a significant over-representation of local rearrangement events was identified in ALS samples from the motor cortex (p=0.0206), cervical spinal cord (p=0.0382), lumbar spinal cord (p=8.997e-04), frontal cortex (p=0.5.256e-05), and cerebellum (p=5.367e-06). Not close proximity gene fusion events were significantly enriched in ALS samples from the cerebellum (p=0.0055). Neighbor gene fusion events were significantly enriched in the ALS samples from the cervical spinal cord (p=0.0021), lumbar spinal cord (p=0.0080), frontal cortex (p=0.0064), temporal cortex (p=2.570e-04), hippocampus (p=0.0155), and cerebellum (p=0.0241). Finally, overlapping neighbor gene fusion events were significantly enriched in ALS samples from the motor cortex (p=0.0458) and frontal cortex (p=0.0222). No enrichment analyses were performed in the medial motor cortex, lateral motor cortex, thoracic spinal cord, sensory cortex, or occipital cortex as there were too few (n<10) tissue samples from controls. *<0.05; **<0.01; *** p<0.001.

FIG. 5. Proportion of gene fusions of each subtype per chromosome. The proportion of breakpoint-unique gene fusions in both ALS and control samples encompassed by each chromosome based on subtypes which included both intra-chromosomal fusions (local rearrangements, neighbor fusions, overlapping neighbor, and not close proximity) and inter-chromosomal fusions.

FIGS. 6A-D. Characterization of the breakpoint-unique gene fusions and their subtypes in ALS and control samples subdivided by capture library preparation method. The breakpoint-unique gene fusions in ALS and control samples subdivided by capture library preparation method were compared to determine the distribution of (A) intra-chromosomal and inter-chromosomal gene fusions, (B) intra-chromosomal gene fusion subtypes, (C) fusion events per sample based on the chromosome(s) involved, and (D) the proportion of fusion events per chromosome(s) involved in the gene fusions corrected for total number of genes located on the chromosome (Ensembl, release 106).

FIGS. 7A-B. Breakpoint-unique gene fusions carried in ALS and control samples subdivided by capture library preparation method. The distribution of breakpoint-unique gene fusions carried per sample was compared between ALS and control samples using the Welch's t-test, both independent of sample tissue source and within each individual tissue source. (A) ALS samples prepared with the automated library preparation method carried significantly more breakpoint-unique gene fusions (mean=12.18, sd=4.72) than control samples (mean=9.41, sd=3.41), but not ALS samples prepared with the manual library preparation method (ALS samples: mean=18.43, sd=7.72; control samples: mean=18.91, sd=8.82). (B) Significantly more breakpoint-unique gene fusions were carried by ALS samples compared to controls prepared with the automated library preparation method in the motor cortex (p=0.0457, cervical spinal cord (p=3.972e-06), lumbar spinal cord (p=5.523e-08), frontal cortex (p=4.221e-08), temporal cortex (p=1.839e-4), hippocampus (p=0.0056), and cerebellum (p=2.625e-4). Significantly fewer breakpoint-unique gene fusions were carried by ALS samples compared to controls prepared with the manual library preparation method in the motor cortex (p=0.0314). No statistical comparisons were performed in the thoracic spinal cord, sensory cortex, or occipital cortex as there were too few (n<10) tissue samples from controls. *<0.05; **<0.01; *** p<0.001.

FIGS. 8A-B. Enrichment of breakpoint-unique gene fusions carried by ALS and control samples subdivided by capture library preparation method. Welch's t-test was used to compare the number of breakpoint-unique gene fusions of each subtype carried by each ALS and control sample both independent of sample tissue source and within each individual tissue source. (A) There was a significant enrichment of the intra chromosomal subtypes local rearrangements, not close proximity fusions, and neighbor fusions in ALS samples compared to control samples prepared using automated capture library preparation methods. There was a significant enrichment of the intra chromosomal subtypes local rearrangements and not close proximity fusions in ALS samples compared to control samples prepared using manual capture library preparation methods. (B) Following subgrouping of gene fusion based on the tissue source of the sample in which they were identified, a significant over-representation of local rearrangement events was identified in ALS samples from the cervical spinal cord (p=0.0018), lumbar spinal cord (p=0.0017), frontal cortex (p=3.672e-06), and cerebellum (p=0.0024) in ALS samples compared to control samples prepared with automated methods. Not close proximity gene fusion events were significantly enriched in ALS samples prepared with automated methods from the cervical spinal cord (p=0.0219), lumbar spinal cord (p=8.637e-04), frontal cortex (p=0.0170), and cerebellum (p=0.0075). Neighbor gene fusion events were significantly enriched in the ALS samples prepared with automated methods from the cervical spinal cord (p=4.614e-06), lumbar spinal cord (p=4.543e-05), frontal cortex (p=1.130e-06), temporal cortex (p=9.118e-04), hippocampus (p=0.0155), and cerebellum (p=0.0477). Overlapping neighbor gene fusion events were significantly enriched in ALS samples prepared with automated methods from the motor cortex (p=0.0146). Local rearrangement gene fusion events were significantly enriched in ALS samples prepared with manual methods from the cerebellum (p=0.0381). No enrichment analyses were performed in the thoracic spinal cord, sensory cortex, or occipital cortex as there were too few (n<10) tissue samples from controls. *<0.05; **<0.01; *** p<0.001.

FIG. 9. Distribution of tissues across ALS and control samples subdivided by capture library preparation method. Several brain regions as well as spinal cord regions were collected per individual ALS and control.

FIG. 10. Proportion of gene fusions of each subtype per chromosome. The proportion of breakpoint-unique gene fusions in both ALS and control samples subdivided by capture library preparation method encompassed by each chromosome based on subtypes which included both intra-chromosomal fusions (local rearrangements, neighbor fusions, overlapping neighbor, and not close proximity) and inter-chromosomal fusions.

FIGS. 11A-B. Effects of expression of YAF2-RYBP fusion protein. (A) Expression of a recombinant YAF2-RYBP fusion protein significantly decreased cell viability in transfected SH-SY5Y cells. (B) Expression of the recombinant YAF2-RYBP fusion protein reduced H2AK119ub1 levels in SH-SY5Y cells.

FIGS. 12A-B. YAF2 and RYBP interacted with polycomb complex proteins Ezh2, RING1A and H3K27me₃ in SH-SY5Y cells. Binding of endogenous RYBP and YAF2 to PRC proteins was demonstrated in SH-SY5Y cells by immunoprecipitation using antibodies to the proteins shown above the blot, followed by immunoblotting using antibodies to RYBP (12A) or YAF2 (12B).

### DETAILED DESCRIPTION

One major potential genetic cause of ALS may be structural variants, such as deletions, duplications, insertions, inversions, and translocations, which have not been systematically examined in ALS. A recent analysis of known ALS-causing genes demonstrated a role for structural variants in this subset of genes.⁶ Specifically, genomic structural variants in C9orf72, valosin-containing protein (VCP) and Erb-B4 receptor tyrosine kinase 4 (ERBB4) genes were shown to modify ALS risk, age, and site of onset as well as progression and survival, highlighting the role of structural variants in ALS pathogenesis.⁶ Similarly, repeat expansions, which are one type of structural variation, in the C9orf72 gene as well as the medium CAG repeat in the ataxin 2 (ATXN2) gene can cause ALS.⁴ Therefore, we hypothesized that a systematic, genome-wide analysis might reveal additional loci where structural variants contribute to the risk of ALS.

Recent studies have demonstrated that genomic instability, mostly due to alterations in DNA damage repair (DRR), may be associated with ALS pathogenesis⁷⁻¹⁰. Of interest, DDR is now considered to be a unifying mechanism underlying neurodegenerative disorders¹¹ and DNA damage is increased and accumulates in the aging brain.¹² In ALS, dysfunction in the DDR mechanism, caused by endogenous sources such as reactive oxygen species¹⁰ or the inability for neurons to recognize or repair DNA damage¹³⁻¹⁴ can trigger onset or worsen disease progression. This has been demonstrated in animal models of ALS as well as by the accumulation of DNA damage in induced-pluripotent stem cell (iPSC)-derived motor neurons and ALS post-mortem brain and spinal cord samples.^{5, 7, 14} Importantly, ALS-associated genes such as SOD1, TARDBP, FUS and (9orf72, are involved in DDR. Specifically, SOD1 can alter DDR mechanism through regulation of transcription, while TARDBP and FUS maintain the balance between single- and double-strand break repair. Lastly, the G₄C₂ repeat expansion in C9orf72 impairs ataxia-telangiectasia mutated (ATM) signaling,⁷ which is critical for the activation of the DNA damage checkpoint during the cell cycle. Together, these findings demonstrate that alterations in genomic stability and DDR occur and may underlie ALS pathogenesis.

While genomic instability includes amplification, translocation, deletion, and inversion events in the genome,¹³ it can also result in gene fusions. Gene fusions are formed when two independent genes become juxtaposed due to structural rearrangements, such as translocations, deletions, and inversions.¹⁵⁻¹⁶ Historically, gene fusions are associated with cancers¹⁷ and cause pathogenesis by either gain or loss of function.¹⁸ Focusing on fusion events in cancer has significantly improved many aspects of clinical care, such as in their use as biomarkers to stratify patients, predict relapse, monitor disease post-treatment, and identify molecular subtypes of cancers.¹⁹⁻²⁰ Importantly, fusion transcripts/proteins are also promising therapeutic targets.²¹⁻²²

This study leveraged RNA-Seq data from Target ALS and the NYGC ALS Consortium. Reported herein for the first time is the presence of gene fusion events in ALS from several brain regions as well as spinal cord. Most fusions were intra-chromosomal events between neighboring genes and there was a significantly greater average number of breakpoint-unique gene fusion events identified per ALS sample compared to controls. Although fusion events were present in nearly all brain and spinal cord samples from both ALS and controls, they were significantly enriched in specific regions, such as cervical and lumbar spinal cord, frontal cortex, temporal cortex, hippocampus, and cerebellum in ALS compared to controls. Statistical comparisons could not be performed for the thoracic spinal cord, sensory cortex, or occipital cortex as there were too few tissue samples from controls. Lastly, specific gene fusions with a significant burden in ALS are highlighted, including rare events that were absent from both known fusion cancer databases and from control samples in our cohort. Together, these findings demonstrate an enrichment of gene fusions that are unique to ALS, suggesting their potential involvement in the genetic etiology of the disease.

The overrepresentation of intra-chromosomal gene fusions in both ALS and control samples was consistent with trends that have been observed in several cancers, such as epithelial and prostate.³¹⁻³² Additionally, recent analysis of human cortex from healthy individuals revealed that most fusion events were formed from genes on the same chromosome.³³ Although the intra-chromosomal gene fusions were also subtyped based on the proximity of the genes involved in the event, ALS samples were found to be significantly enriched for all four intra-chromosomal subtypes, including (1) local rearrangements, (2) not close proximity fusions, (3) neighbors, and (4) overlapping neighbors. Specific gene fusion pairs were also identified within the tissues demonstrating significant enrichment of intra-chromosomal fusion subtypes. In some cases, these fusions demonstrated significant burden in ALS samples across all tissue samples, such as the local rearrangement AC006427.2-TAPT1-AS1, whereas other fusions demonstrated significant burden in ALS samples specifically in certain tissue types, such as the neighbor fusion in the cervical spinal cord samples, PAMR1-SLC1A2, which was found to have a significant burden in ALS samples (56/265) compared to controls (0/45). The top 90 fusions present in ALS samples but absent in controls are shown in Table 3, below.

Previously, gene fusions were largely detected using fluorescence in situ hybridization and quantitative real-time polymerase chain reaction; however, these methods do not allow for an agnostic screen of all potential fusion events. Rather, these methods specifically target known gene fusions.³⁴ In contrast, RNA-Seq has proven to be an efficient method for detecting gene fusions across the entire transcriptome. Recently, 23 RNA-Seq fusion detection methods were compared to examine accuracy as well as relative computational speed, and the STAR-Fusion algorithm was considered a top performer in both respects.²³ In the present study, algorithms were chosen to minimize the possibility of F positive findings. For example, STAR-Fusion employs several filtration steps, including referencing against gene fusion databases from control populations to ignore fusions expected in healthy people.

The gene fusions described herein may contribute to the development of ALS as has been shown in oncology. Fusion genes are well-defined oncogenic drivers in several different types of cancer, demonstrating their potential for reprogramming of normal cellular function. Indeed, fusion events can lead to either gain or loss of function, causing overexpressed, constitutively active, or truncated products.³⁶ ALS is approximately 50% heritable,³ yet the known ALS causing genes are present in less than 15% of patients. It is possible that some fusion events will explain the missing heritability. Some of the events that we identified are present at lower frequency in people without ALS. These events may be causes of ALS but incompletely penetrant. The present work also identified many events that were previously unknown in cancer or healthy individuals. Most of these did not reach statistical significance, but that may reflect the relatively small number of control samples currently available.

The exact mechanisms leading to fusion events are not completely understood. Alterations in both DDR and RNA metabolism have been implicated as potential mechanisms leading to fusion events.¹¹ Specifically, defects in DDR mechanisms have been described in motor neurons derived from people living with ALS and were associated with faster disease progression.³⁷ Therefore, it is possible that alterations in DDR may be one possible mechanism leading to intra-chromosomal fusion events in ALS. In this study, many fusions unique to ALS involved non-coding genes and non-coding RNA (ncRNAs). Therefore, alterations in RNA splicing could also account for some of the intra-chromosomal fusions reported in this study, perhaps through the contribution of another rare phenomenon, trans-splicing.

The identification and characterization of fusion events in cancer has notably improved diagnosis, prognosis and treatment.^{17,21} For example, the CLDN18-ARHGAP fusion is an important diagnostic and prognostic risk factor for gastric cancer,³⁸ while the DNAJB1-PRKACA chimeric transcript contributes to the pathogenesis of the fibrolamellar carcinoma (FC).^{19,39} Gene fusion events have been also described in brain cancers with several targetable fusion events in malignant gliomas^{22, 28} and neuroblastomas.⁴⁰ Additionally, the identification of gene fusions has recently been applied to constitutional diseases, specifically in a variety of rare, undiagnosed phenotypes, which was found to result in improved diagnoses as well.⁴¹⁻⁴² As ALS is a multifactorial and heterogenous neurodegenerative disease arising from a combination of genetic and environmental factors, the enrichment of gene fusions identified herein suggests a role for structural genomic anomalies in ALS risk, onset or progression.

### Methods of Diagnosis and Determining Risk

Provided herein are methods that can be used for diagnosing ALS, or determining risk of developing ALS. The methods include detection of a fusion described herein, or a plurality of fusions described herein, e.g., as shown in Tables 2-7. The methods can include detecting genomic fusions, fused transcripts, or fusion proteins (where proteins are produced) as described herein. The methods can include obtaining a sample from a subject, and evaluating the presence of a fusion described herein in the sample. In some embodiments, the methods include comparing a sequence obtained from the sample with a reference sequence, e.g., a control sequence that represents a normal sequence, e.g., in an unaffected subject without a fusion present, and/or a disease reference that represents a sequence of a fusion described herein. In some embodiments, the reference sequence is a human sequence present in a database, e.g., NCBI GenBank RefSeq database (searchable and available online at ncbi.nlm.nih.gov/refseq), e.g., in human reference genome GRCh38.p14 (See, e.g., O'Leary et al., Nucleic Acids Res. 2016 44 (D1): D733-45). In some embodiments, the methods include detecting presence of a fusion described herein without determining the full sequence of the fusion.

In some embodiments, the methods can also include detecting the presence or levels of neurofilament light chain (NFL) in serum or CSF (see, e.g., Verde et al., Front Neurosci. 2021 Jun. 21; 15:679199; Gaiani et al., JAMA Neurol. 2017 May 1; 74 (5): 525-53; Falzone et al., Neural Regen Res. 2021 October; 16 (10): 1985-1991), or the presence of other genetic alterations associated with ALS, e.g., mutations in a gene associated with ALS such as Superoxide dismutase 1 (SOD1), Alsin (ALS2), SETX, SPG11, FUS, VABP, Angiogenin (ANG), TAR DNA-binding protein (TARDBP), FIG4, OPTN, ATXN2, ubiquilin 2 (UBQLN2), C9ORF72 (e.g., a hexanucleotide (GGGGCC) repeat expansion in C9ORF72), SIGMAR1, or Profilin 1 (PFN1).

As used herein the term “sample”, when referring to the material to be tested for the presence of a biological marker using the method of the invention, includes inter alia tissue, whole blood, plasma, serum, saliva, exosome or exosome-like microvesicles (U.S. Pat. No. 8,901,284), lymph, or cerebrospinal fluid (CSF). In some embodiments, the sample is serum or CSF. Various methods are well known within the art for the identification and/or isolation and/or purification of a sequence from a sample. For example, nucleic acids contained in the sample can be isolated according to known methods, for example using lytic enzymes, chemical solutions, or isolated by nucleic acid-binding resins following the manufacturer's instructions.

The presence and/or level of a protein can be evaluated using methods known in the art, e.g., using standard electrophoretic and quantitative immunoassay methods for proteins, including but not limited to, Western blot; enzyme linked immunosorbent assay (ELISA); biotin/avidin type assays; protein array detection; radio-immunoassay; immunohistochemistry (IHC); immune-precipitation assay; FACS (fluorescent activated cell sorting); and mass spectrometry (Kim (2010) Am J Clin Pathol 134:157-162; Yasun (2012) Anal Chem 84 (14): 6008-6015; Brody (2010) Expert Rev Mol Diagn 10 (8): 1013-1022; Philips (2014) PLOS One 9 (3):e90226; Pfaffe (2011) Clin Chem 57 (5): 675-687), e.g., using antibodies that bind specifically to a fusion described herein. The methods typically include revealing labels such as fluorescent, chemiluminescent, radioactive, and enzymatic or dye molecules that provide a signal either directly or indirectly. As used herein, the term “label” refers to the coupling (i.e. physically linkage) of a detectable substance, such as a radioactive agent or fluorophore (e.g. phycoerythrin (PE) or indocyanine (Cy5), to an antibody or probe, as well as indirect labeling of the probe or antibody (e.g. horseradish peroxidase, HRP) by reactivity with a detectable substance.

In some embodiments, an ELISA method may be used, wherein the wells of a microtiter plate are coated with an antibody against which the protein is to be tested. The sample containing or suspected of containing the biological marker is then applied to the wells. After a sufficient amount of time, during which antibody-antigen complexes would have formed, the plate is washed to remove any unbound moieties, and a detectably labelled molecule is added. Again, after a sufficient period of incubation, the plate is washed to remove any excess, unbound molecules, and the presence of the labeled molecule is determined using methods known in the art. Variations of the ELISA method, such as the competitive ELISA or competition assay, and sandwich ELISA, may also be used, as these are well-known to those skilled in the art.

In some embodiments, an IHC method may be used. IHC provides a method of detecting a fusion described herein in situ. The presence and exact cellular location of the fusion protein can be detected. Typically a sample is fixed with formalin or paraformaldehyde, embedded in paraffin, and cut into sections for staining and subsequent inspection by confocal microscopy. Current methods of IHC use either direct or indirect labelling. The sample may also be inspected by fluorescent microscopy when immunofluorescence (IF) is performed, as a variation to IHC.

Mass spectrometry, and particularly matrix-assisted laser desorption/ionization mass spectrometry (MALDI-MS) and surface-enhanced laser desorption/ionization mass spectrometry (SELDI-MS), is useful for the detection of a fusion described herein. (See U.S. Pat. Nos. 5,118,937; 5,045,694; 5,719,060; 6,225,047)

The presence and/or level of a fusion nucleic acid (e.g., gDNA or mRNA) as described herein can be evaluated using methods known in the art. For example, the presence of a fusion in gDNA can be assayed, e.g., using polymerase chain reaction (PCR), reverse transcriptase polymerase chain reaction (RT-PCR), quantitative or semi-quantitative real-time RT-PCR, droplet digital PCR (ddPCR), digital PCR, e.g., BEAMing ((Beads, Emulsion, Amplification, Magnetics) Diehl (2006) Nat Methods 3:551-559). The presence of RNA fusions can be assayed, e.g., using RNA-Seq; RNAse protection assay; Northern blot. DNA or RNA fusions can be assayed, e.g., using nucleic acid sequencing (e.g., Sanger, pyrosequencing, NextGeneration Sequencing, or Long Read Sequencing). See, e.g., Lehninger Biochemistry (Worth Publishers, Inc., current addition; Sambrook, et al, Molecular Cloning: A Laboratory Manual (3rd Edition, 2001); Bernard (2002) Clin Chem 48 (8): 1178-1185; Miranda (2010) Kidney International 78:191-199; Bianchi (2011) EMBO Mol Med 3:495-503; Taylor (2013) Front. Genet. 4:142; Yang (2014) PLOS One 9 (11):e110641); Nordstrom (2000) Biotechnol. Appl. Biochem. 31 (2): 107-112; Ahmadian (2000) Anal Biochem 280:103-110.

In some embodiments, high throughput methods, e.g., protein or gene chips as are known in the art (see, e.g., Ch. 12, Genomics, in Griffiths et al., Eds. Modern genetic Analysis, 1999, W. H. Freeman and Company; Ekins and Chu, Trends in Biotechnology, 1999, 17:217-218; MacBeath and Schreiber, Science 2000, 289 (5485): 1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of a fusion nucleic acid or protein as described herein. For example, antibodies or oligonucleotide probes that bind specifically to a fusion junction sequence can be used.

Measurement of a fusion as described herein can be direct or indirect. For example, the abundance levels of a fusion nucleic acid as described herein can be directly quantitated. Alternatively, the amount of a fusion nucleic acid can be determined indirectly by measuring abundance levels of cDNA, amplified RNAs or DNAs, or by measuring quantities or activities of RNAs, or other molecules that are indicative of the expression level of the fusion.

In some embodiments a technique suitable for the detection of alterations in the structure or sequence of nucleic acids, such as the presence of deletions, amplifications, or substitutions, can be used for the detection of biomarkers of this invention. For example, PCR can be used to detect fusions, either by comparing amplicon length (wherein the presence of an amplicon that is longer or shorter than expected if there was no fusion event) or by sequencing the amplicons (wherein the fusion can be directly detected by comparing the amplicon sequence to a reference or control sequence that represents a wild type sequence in the absence of a fusion event).

As one example, RNA-Seq can be used to detect the presence of a fusion nucleic acid as described herein. The template RNA (optionally enriched by depletion of unwanted sequences or by isolation of RNA from exosomes) is contacted with a set of probes that bind to the target sequence and reverse transcribed into cDNA, followed by next-generation sequencing. See, e.g., Hong et al., Journal of Hematology & Oncology 13:166 (2020). In some embodiments, enrichment baits are used that bind to the fusion proteins for target capture (see, e.g., Zhou et al., Hereditas 158:10 (2021)). In some embodiments, long-read sequencing is used, e.g., using a long read sequencer from Pacific Biosciences (PacBio) and Oxford Nanopore Technologies (Nanopore) (see, e.g., Jain et al. Nat Biotechnol. 2018 April; 36 (4): 338-345; Payne et al. Bioinformatics. 2019 Jul. 1; 35 (13): 2193-2198; Stancu et al., Nat Commun. 2017 Nov. 6; 8 (1): 1326; Zhiao and Xianghuo, 2021. Medical Reviews 1 (2): 150-171; Ebbert et al., 2018. Molecular Neurodegeneration 13 (1): 46; Beyter et al., 2021. Nature Genetics 53 (6): 779-786; Levy et al., 2022. Human Genetics and Genomics Advances 3 (1): 100075.

Arrays can be prepared by selecting probes that comprise a polynucleotide sequence that binds to a fusion described herein, and optionally immobilizing such probes to a solid support or surface or to beads. For example, the probes may comprise DNA sequences, RNA sequences, co-polymer sequences of DNA and RNA, DNA and/or RNA analogues, or combinations thereof. The probe sequences can be synthesized either enzymatically in vivo, enzymatically in vitro (e.g. by PCR), or non-enzymatically in vitro.

In some embodiments, presence of a fusion as described herein is detected, and the subject has one or more symptoms associated with ALS, then the subject can be diagnosed with ALS. In some embodiments, the subject has no overt signs or symptoms of ALS, but the presence of a fusion as described herein, then the subject has an increased risk of developing ALS. In some embodiments, once it has been determined that a person has ALS, or has an increased risk of developing ALS, then a treatment, e.g., as known in the art or as described herein, can be administered. In some embodiments, the subject is suspected of having ALS, e.g., as a result of loss of function or gradual progressive weakness without pain in one or more regions of the body, without changes in the ability to feel, where no other cause is evident. In some embodiments, the subject has upper motor neuron (UMN) and/or lower motor neuron (LMN) signs. UMN signs are mild weakness, spasticity, and abnormally brisk reflexes; LMN signs are progressive weakness, wasting, and loss of reflexes and muscle tone. In some embodiments, the methods include further diagnostic evaluation of subject, e.g., using the revised El Escorial criteria (Brooks et al., J Neurol Sci. 1994 July; 124 Suppl: 96-107), Awaji criteria (de Carvlaho et al., Clin Neurophysiol. 2008 March; 119 (3): 497-503), or World Federation of Neurology (WFN) criteria (Brooks et al., Amyotroph Lateral Scler Other Motor Neuron Disord. 2000 December; 1 (5): 293-9).

In some embodiments, once presence of a fusion as described herein is detected, the subject can be further evaluated using methods known in the art to confirm a diagnosis of ALS. Such methods can include assaying the presence or levels of neurofilament light chain (NFL) in serum or CSF; electrodiagnostic tests, including electomyography (EMG) and nerve conduction velocity (NCV); blood and urine studies, including high resolution serum protein electrophoresis, thyroid and parathyroid hormone levels and 24-hour urine collection for heavy metals; spinal tap; X-rays, including magnetic resonance imaging (MRI); myelogram of cervical spine; muscle and/or nerve biopsy; and neurological examination.

In some embodiments, once presence of a fusion as described herein is detected, the subject can be treated, e.g., with a treatment for ALS, e.g., riluzole, sodium phenylbutyrate-taurursodiol, or edaravone; see also Feldman et al., Lancet. 2022 Oct. 15; 400 (10360): 1363-1380. The methods can also be used to stratify subjects in a clinical trial, e.g., to associate presence of a particular fusion event with response or non-response to a candidate treatment.

Also provided herein are kits for use in the present methods, e.g., comprising oligonucleotides that bind to each of the genes in the fusions, e.g., to YAF2 and RYBP, and reagents for detecting them. Optionally the oligonucleotides comprise barcodes or are detectably labeled, e.g., with a fluorescent or other label.

### EXAMPLES

The invention is further described in the following examples, which do not limit the scope of the invention described in the claims.

### Methods

The following materials and methods were used in the Examples below.

### Source of RNA-Seq Data

All RNA-Seq data used in this paper were previously generated by Target ALS and the New York Genome Center (NYGC) ALS Consortium and were shared with us under a collaborative research agreement. These data consist of RNA-Seq from the motor cortex (including medial, lateral, and unspecified), cervical spinal cord, thoracic spinal cord, lumbar spinal cord, frontal cortex, temporal cortex, occipital cortex, hippocampus, and cerebellum of ALS and control individuals. Information on the sample preparation, sequencing and quality control can be obtained from the Center for Genomics of Neurodegenerative Disease (CGND) at the NYGC. Importantly, quality control of the data accounted for high-fidelity base predictions, GC content, total read count, percent of duplicate reads, percent of rRNA, and potential sample contamination.

Determining Gene Fusion Events from Bulk RNA-Seq

Gene fusion predictions were identified using STAR-Fusion v1.10.0 with default settings.²³ STAR-Fusion uses the RNA-Seq read aligner, STAR,²⁴⁻²⁶ to align reads with command-line flags optimized for fusion detection. Briefly, chimeric reads from STAR alignment were isolated to begin fusion prediction. Chimeric reads occur when either (1) a portion of a read aligns to one gene and another portion of the same read aligns to a different gene (split) or when (2) each end of a paired read set aligns to different genes (spanning). Using these chimeric reads, STAR-Fusion uses all-vs-all blastn to remove F positive chimeric alignments that are caused by sequence similarity. Following all-vs-all blastn filtering, the remaining set of reads was considered for gene fusions. Candidate gene fusion pairs with only one split read or one spanning read pair were discarded. Using the Duplicated Genes Database, fusions involving genes that are likely paralogs of each other were also removed as these predictions may have been due to sequence similarity. If certain genes were found to have over 10 other genes as potential fusion partners, these genes were removed from consideration as being “promiscuous”. Recurrent fusions found in healthy RNA-Seq datasets, such as the Genotype-Tissue Expression project (GTEx), Illumina Human Body Map and 1000 Genomes RNA-Seq, were removed to limit the possibility of F positives. Lastly, fusion candidates were filtered based on the number of reads providing evidence for the event. This was done using fusion fragments per million total RNA-Seq fragments (FFPM). Fusions with FFPM less than 0.1 (one evidence fragment per ten million total reads) were discarded as this ratio corresponds to the 99th percentile of ratios identified for fusions in GTEx samples. Importantly, within each sample, a specific gene fusion can have multiple high-confidence breakpoints, which denote the base pair for each gene in the pair where the gene either ends or begins. To avoid counting fusions multiple times within the same sample in future analyses, the dataset was filtered to only include the most common breakpoint for each fusion in each sample. Hence, all downstream analyses were done with “breakpoint-unique gene fusions.” All gene fusion events were classified based on the regions involved, first, broadly into inter-chromosomal and intra-chromosomal fusions. The intra-chromosomal fusions were further classified into four subtypes: (1) local rearrangements, which were fusions where the genes are in an unexpected order given the strand of each gene in the pair; (2) not close proximity, which encompassed genes>100 kb apart; (3) neighbors, which were fusions that encompassed genes<100 kb apart and did not show evidence of gene orientation rearrangement; and (4) overlapping neighbors, which encompassed genes whose spans overlapped by at least one base pair.

### Dataset Quality Control

The dataset was filtered by ancestry to avoid any potential confounding factors. Specifically, bulk RNA-Seq samples from patients with greater than 80% European ancestry were kept in the final analysis cohort. Furthermore, principal component analysis (PCA) was used to determine if batch effects existed between samples based on a variety of co-variates, including project, sequencing platform, capture library preparation method, sample tissue of origin, subject ethnicity, and subject sex. The underlying matrix used for this analysis included all samples carrying unique fusion gene pairs found in our analysis cohort and the FFPM metric for that specific fusion and specific sample. Our analysis identified no batch effects when mixing data from both sources, indicating the datasets could be binned for downstream analyses.

### Gene Fusion Enrichment Analysis

The distribution of breakpoint-unique gene fusions carried per sample was compared between ALS and control samples using Welch's t-test. Comparisons were performed both independent of sample tissue source and within each individual tissue source. Welch's t-test was also used to examine the association between specific intra-chromosomal gene fusion subtypes and ALS across all tissues by comparing the number of breakpoint-unique gene fusions of each subtype carried per sample between ALS and control samples. Subsequently, Welch's t-test was used to examine the association between specific intra-chromosomal fusion subtypes and ALS at a tissue-specific level. For all statistical analyses, no statistical comparisons were performed in the thoracic spinal cord, sensory cortex, or occipital cortex as there were too few (n<10) tissue samples from controls.

Following initial enrichment analyses of the full gene fusion dataset, it was determined that multiple library preparation methods were used in the initial RNA sequencing. A portion of the samples were prepared using manual capture library preparation, meaning that a technician performed the library preparation by hand; whereas the remaining samples were prepared using an automated library preparation, which is performed by an automated robotic system, which is now the conventional approach. Although examination of the PCA did not demonstrate any significant batch effects from library preparation method; to be cautious, we subdivided the samples based on their library preparation method, and gene fusion enrichment analyses were repeated to ensure signals of enrichment were not technical artifacts driven by the methodology.

### Individual Gene Fusion Burden Analysis

We determined whether each pair of genes encompassed by a fusion, hereafter referred to as a “gene fusion pair”, found in our cohort was observed at a greater or lesser burden in ALS than control samples using Fisher's exact test based on the counts of samples with or without the gene fusion pair. The test was first done using the sum of all counts observed across all tissues. The results were also filtered to include only the significant gene fusions absent from cancer fusion databases and absent from the control samples in our cohort, hereafter referred to as “rare gene fusions.” Lastly, we performed burden analysis using Fisher's exact testing on gene fusion pairs of each intra-chromosomal subtype that was significantly enriched in specific tissues. In this way were able to determine whether individual gene fusion pairs may be driving the signals of enrichment observed in the previous gene fusion enrichment analysis. The gene fusion pairs of each subtype that were identified in significant tissues were first binned, and a Fisher's exact test was run for each intra-chromosomal subtype, followed by individual Fisher's exact test on gene fusion pairs identified in each individual tissue for each intra-chromosomal subtype that demonstrated significant enrichment.

### Data Visualization and Statistical Analysis

Statistical analyses were performed using R statistical software 4.1.1 (R Core Team, 2014) in RStudio 1.4.1717. Data visualization was performed using the ggplot2 R package (v3.3.5).²⁷ For all individual gene fusion pair burden analyses, corrected p-values were calculated using Bonferroni corrections based on the total number of breakpoint-unique fusions observed within the respective tissue(s) and significance was measured at an alpha-level of p<0.05. Circos plots were generated by using the shinyCircos²⁸ web interface (venyao.xyz/shinyCircos/). PCA was conducted with default flags in scikit-learn v1.029 with Python 3.9.4 using the fit_transform( ) function and PCA biplots were rendered using matplotlib 3.4.2.³⁰

### Study Approval

The study was approved by the Partners Healthcare IRB. Written informed consent was obtained from all participants prior to study enrollment. Post-mortem consent was obtained from the appropriate representative (next of kin or health care proxy) prior to autopsy.

### Example 1. Identification and Classification of Gene Fusion Events from RNA-Seq Datasets

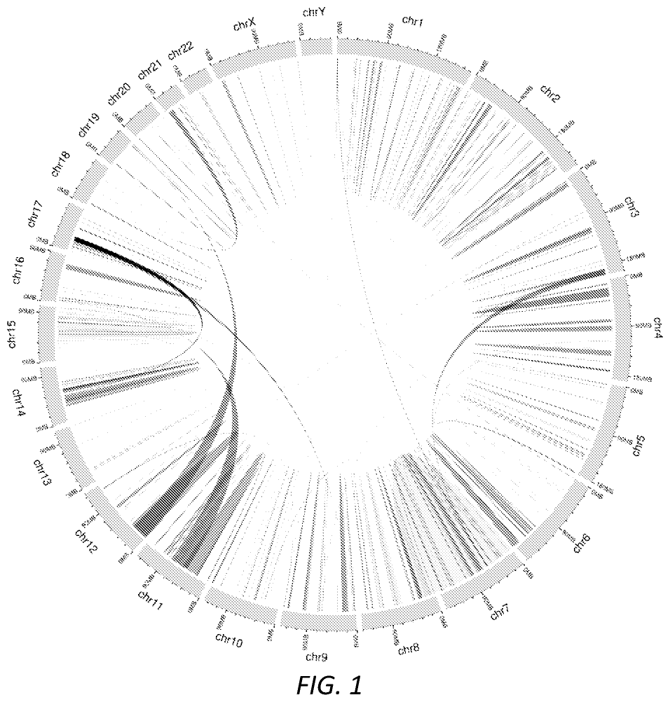

The RNA-Seq datasets from Target ALS and the ALS Consortium consisted of 367 individuals with ALS and 90 controls with several tissue samples collected per individual resulting in a total of 1,542 ALS and 249 control samples (Table 1). In total, 607 unique pairs of genes were observed to form fusions. There was a total of 21,872 breakpoint-unique gene fusions in ALS samples, and a total of 2,780 breakpoint-unique gene fusions in control samples (FIG. 1). To ensure that there were no potential batch effects from project, sequencing platform, capture library preparation method, sample tissue of origin, subject ethnicity, and subject sex we performed a principal component analysis (PCA) on the matrix of fusion fragments per million total RNA-Seq fragments (FFPM) values. The assessed co-variates introduced minimal variance in the gene fusion data.

TABLE 1
Demographics of tissue samples from ALS and controls.

| | ALS | Control |
|---|---|---|
| Total Subjects | 367 | 90 |
| Target ALS Subjects | 177 | 10 |
| ALS Consortium Subjects | 177 | 80 |
| Male:Female | 200:167 | 52:36 |

Ethnicity (self-reported)

| | | |
|---|---|---|
| Hispanic or Latino | 1 | 1 |
| Not Hispanic or Latino | 131 | 8 |
| Ashkenazi Jewish | 1 | 0 |
| Unknown | 227 | 80 |
| White Scottish | 7 | 1 |
| Tissues Samples per Subject (mean; SD) | 4.20 (2.26) | 2.77 (1.61) |
| Tissue Samples | 1542 | 249 |
| Motor Cortex | 373 | 48 |
| Spinal Cord - Cervical | 265 | 45 |
| Spinal Cord - Thoracic | 56 | 5 |
| Spinal Cord - Lumbar | 252 | 41 |
| Frontal Cortex | 248 | 48 |
| Sensory Cortex | 2 | 0 |
| Temporal Cortex | 34 | 25 |
| Occipital Cortex | 64 | 6 |
| Hippocampus | 61 | 13 |
| Cerebellum | 187 | 29 |

Capture Library Preparation Method

| | | |
|---|---|---|
| Automated | 1047 | 203 |
| Manual | 495 | 46 |
| Age at Symptom Onset (mean; SD) | 60.52 (11.32) | N/A |
| Bulbar Onset:Limb Onset:Other/Unknown | 112:218:37 | N/A |

Sex was unknown for two control samples. Age at symptom onset was unknown for 53 ALS samples.

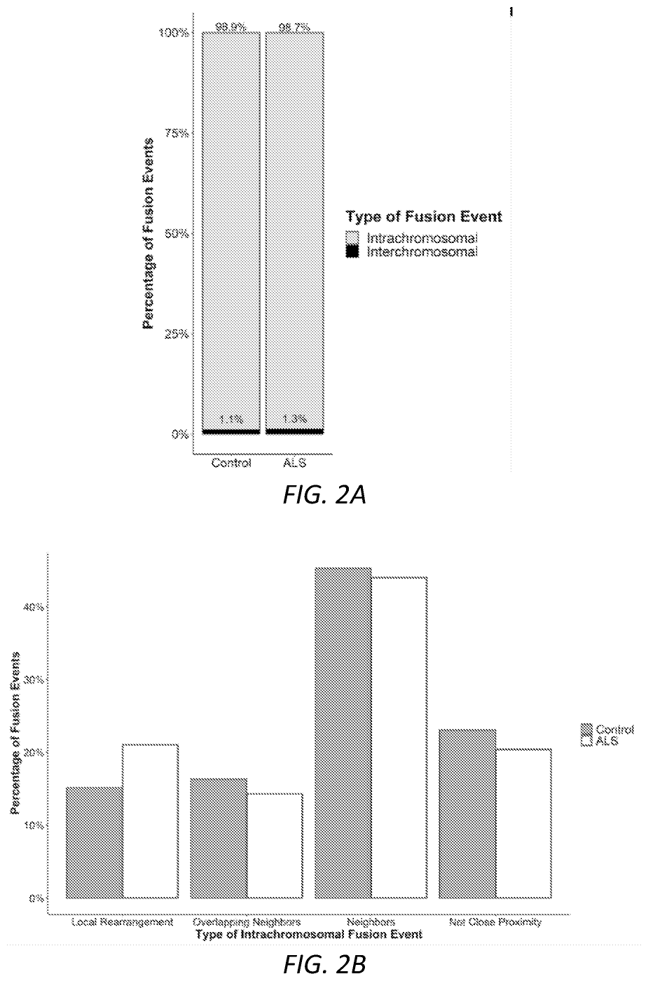

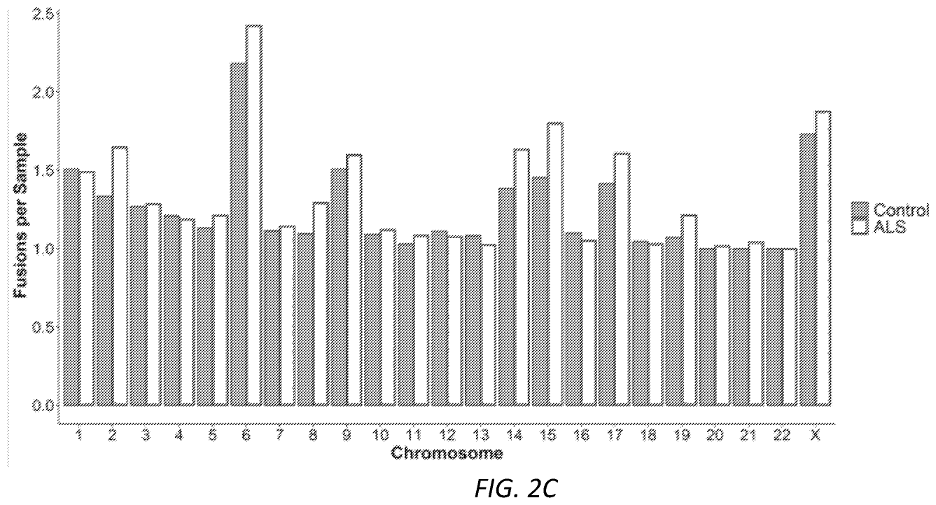

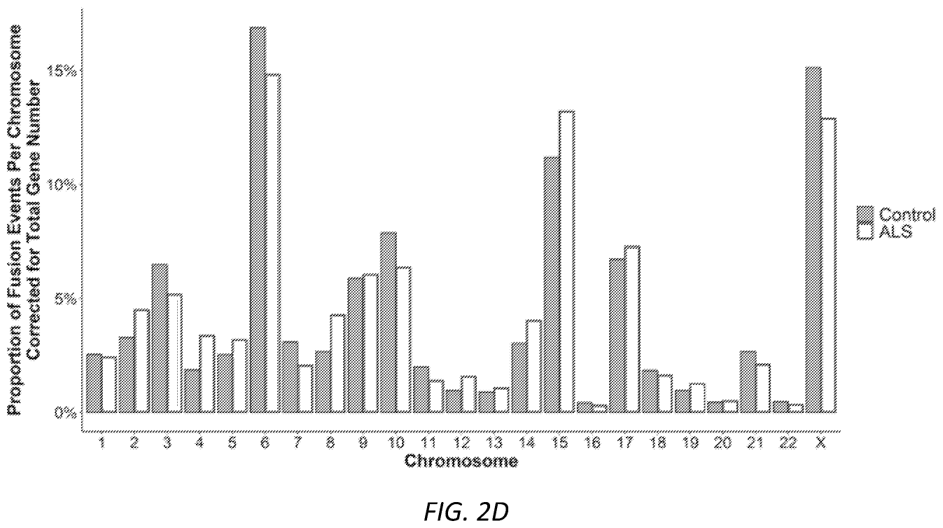

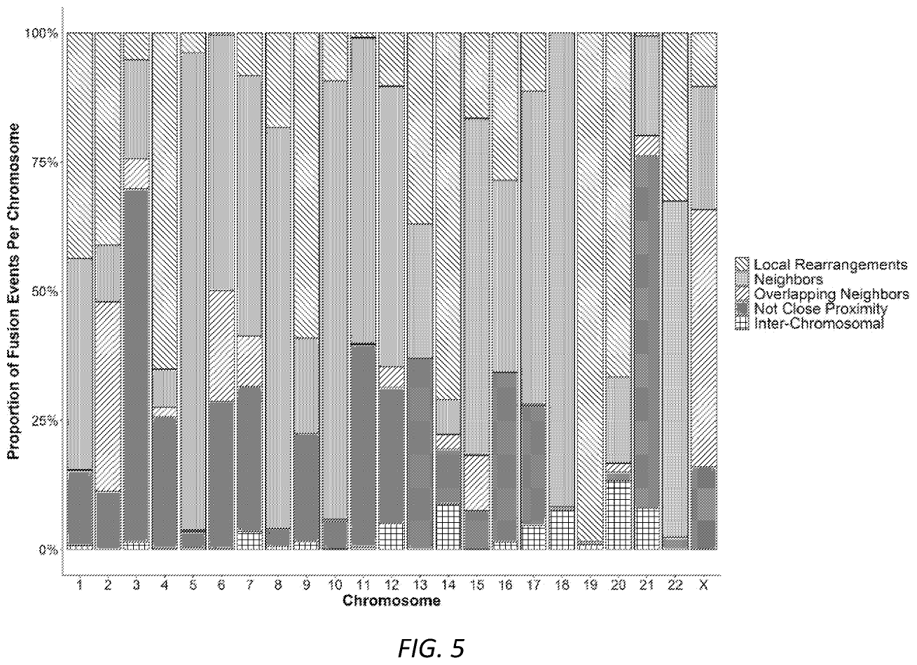

To determine the origin of the gene fusions, we surveyed the proportion of inter-chromosomal versus intra-chromosomal events based on sample condition and found that most fusion events (>98%) were intra-chromosomal in both ALS and controls. We further divided these events into their intra-chromosomal fusion subtypes: local rearrangements, not close proximity fusions, neighbors, and overlapping neighbors (FIG. 2A). Although most fusions were classified as neighbors in both ALS and control samples, we also identified a proportion of events classified as overlapping neighbor, not close proximity or local rearrangements in both ALS and control samples (FIG. 2B). Furthermore, the chromosomes most often involved in the fusion events were chromosomes 6 and X, in both ALS and controls (FIGS. 2C-D). A summary of the different subtypes of gene fusions found per chromosome are displayed in FIG. 5.

### Example 2. Distribution of Gene Fusion Events Between ALS and Controls

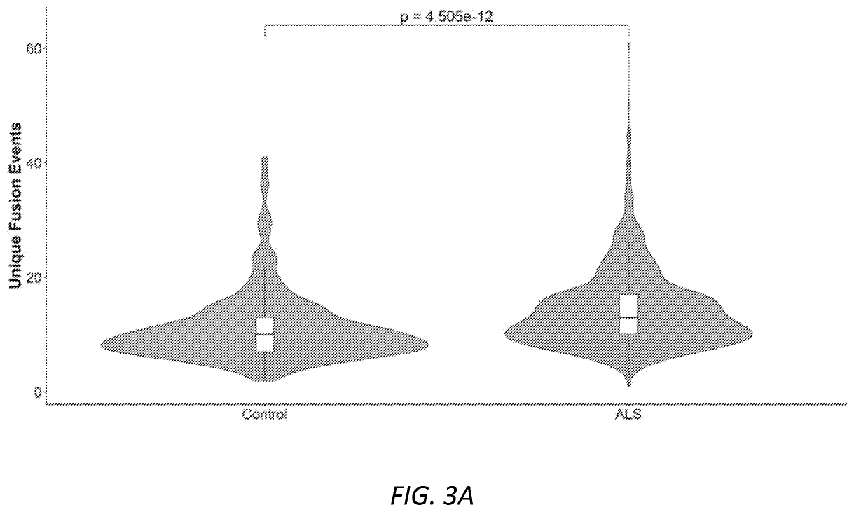

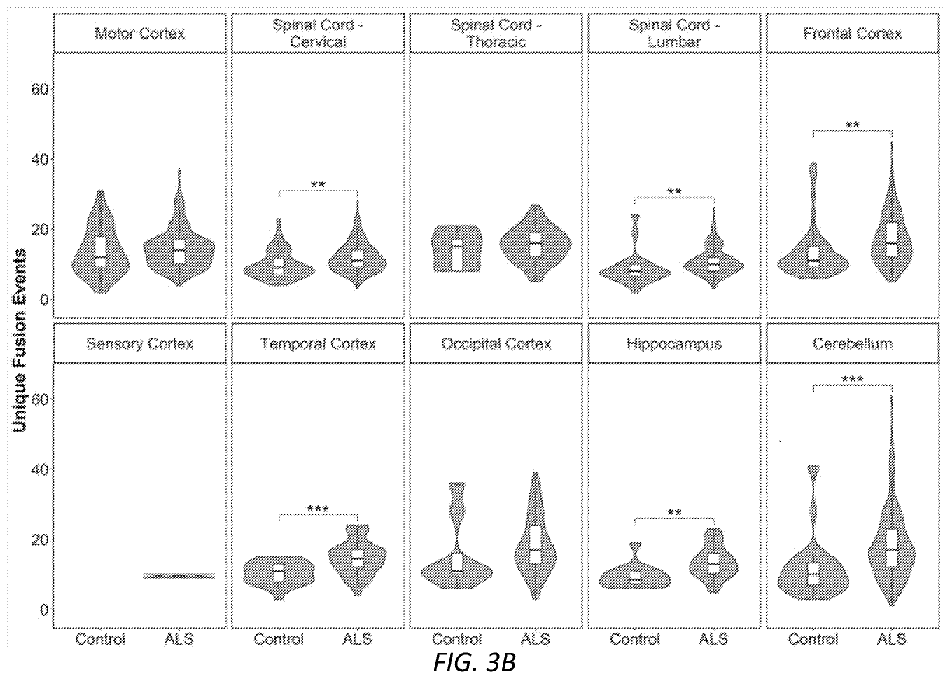

To characterize the distribution of fusions, we compared the number of breakpoint-unique gene fusions carried by each ALS and control sample (FIG. 3A). On average, ALS samples each carried significantly more breakpoint-unique gene fusion events than controls (mean±SD: 14.18±6.54 and 11.16±6.10, respectively; Welch's t-test, p=4.505e-12). In particular, ALS samples each carried significantly more intra-chromosomal gene fusion events than the control samples (mean±SD=14.00±6.45, and 11.04±6.03, respectively; Welch's t-test, p=6.257e-12). However, there was no significant difference between the number of inter-chromosomal gene fusion events carried by each sample from ALS and controls (mean±SD=1.13±0.36, and 1.07±0.26, respectively; Welch's t-test, p=0.2677).

Next, we compared the number of breakpoint-unique gene fusions carried by each ALS and control sample within the individual tissue sources (FIG. 3B). ALS samples had significantly more gene fusion events than controls as measured by Welch's t-test in the following tissues: the cervical spinal cord (p=0.0022), lumbar spinal cord (p=0.0012), frontal cortex (p=0.0011), temporal cortex (p=2.375e-4), hippocampus (p=0.0056), and cerebellum (p=1.393e-4). There were no tissues in which control samples had more gene fusion events than ALS samples.

### Example 3. Enrichment of Intra-Chromosomal Gene Fusion Events

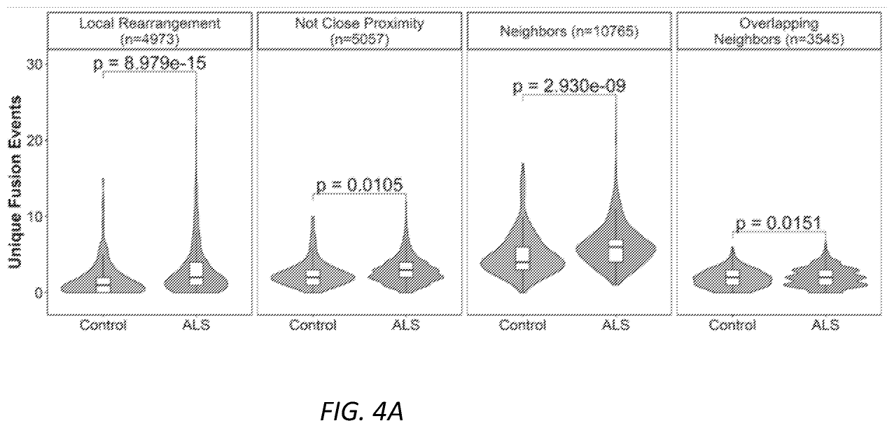

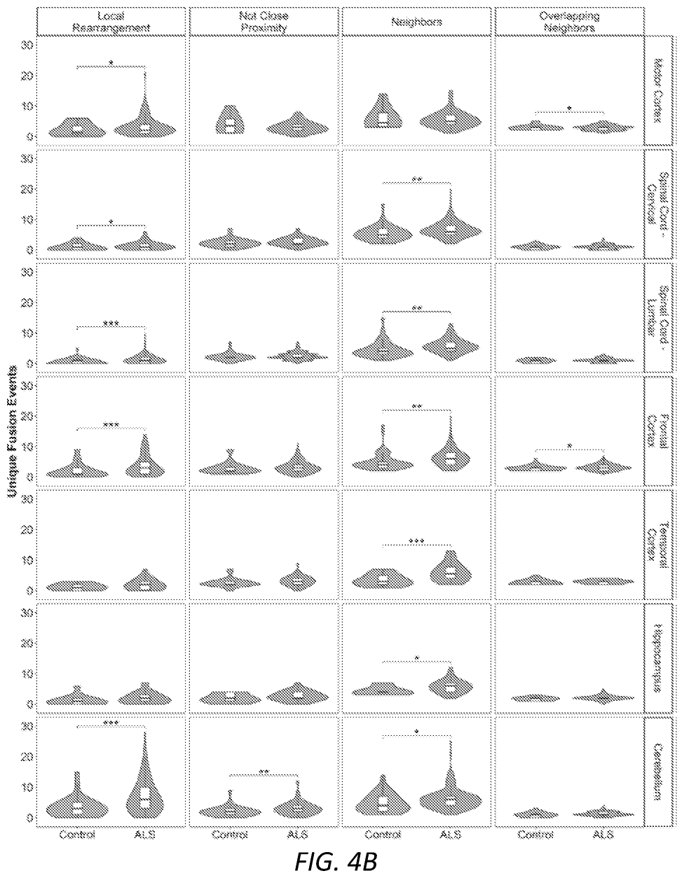

We tested whether ALS samples were enriched for specific subtypes of intra-chromosomal gene fusion events (FIGS. 4A-B). Across samples from all tissues, we identified a significant enrichment of all four intra-chromosomal subtypes in the ALS samples compared to the controls as measured by Welch's t-test: local rearrangements (p=8.979e-15), not close proximity fusions (p=0.0105), neighbor fusions (p=2.930e-09), and overlapping neighbor fusions (p=0.0151).

We carried out the same analysis separately for each tissue (FIG. 4B). Local rearrangement events were significantly over-represented in ALS samples from the motor cortex (p=0.0206), cervical spinal cord (p=0.0382), lumbar spinal cord (p=8.997e-04), frontal cortex (p=5.256e-05), and cerebellum (p=5.367e-06). Not close proximity gene fusion events were significantly enriched in ALS samples from the cerebellum (p=0.0055). Neighbor gene fusion events were significantly enriched in the ALS samples from the cervical spinal cord (p=0.0021), lumbar spinal cord (p=0.0080), frontal cortex (p=0.0064), temporal cortex (p=2.570e-04), hippocampus (p=0.0155), and cerebellum (p=0.0241). Finally, overlapping neighbor gene fusion events were significantly enriched in ALS samples from the motor cortex (p=0.0458) and frontal cortex (p=0.0222).

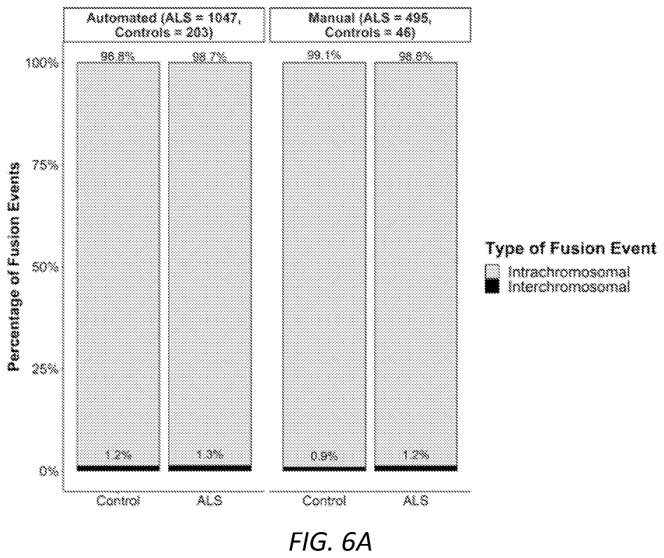

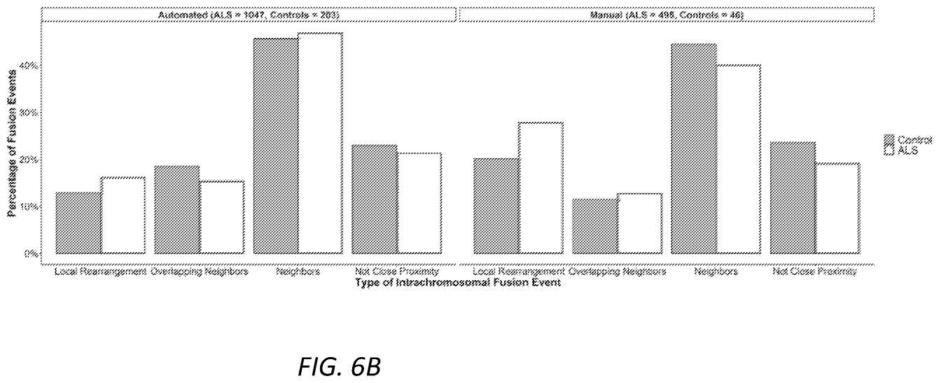

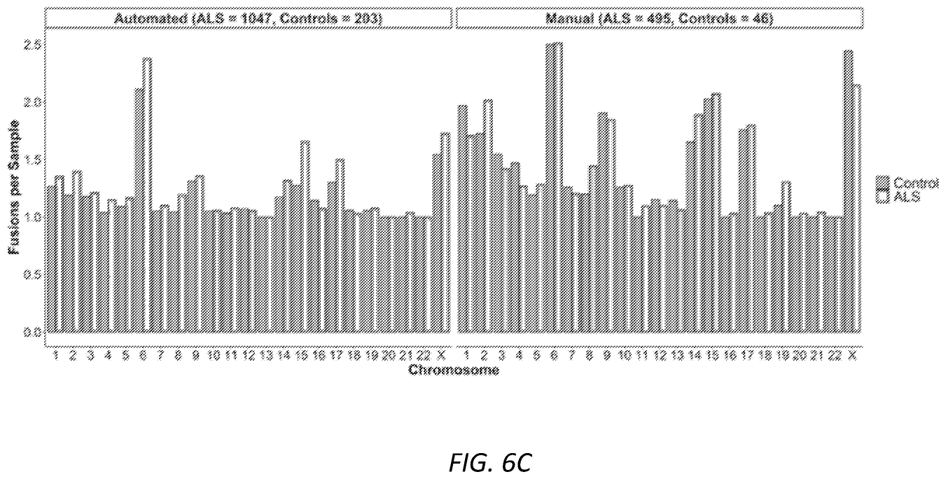

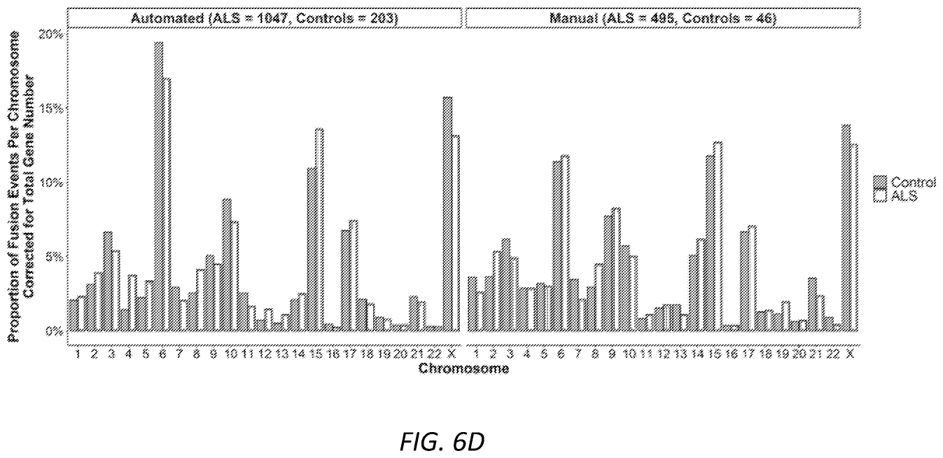

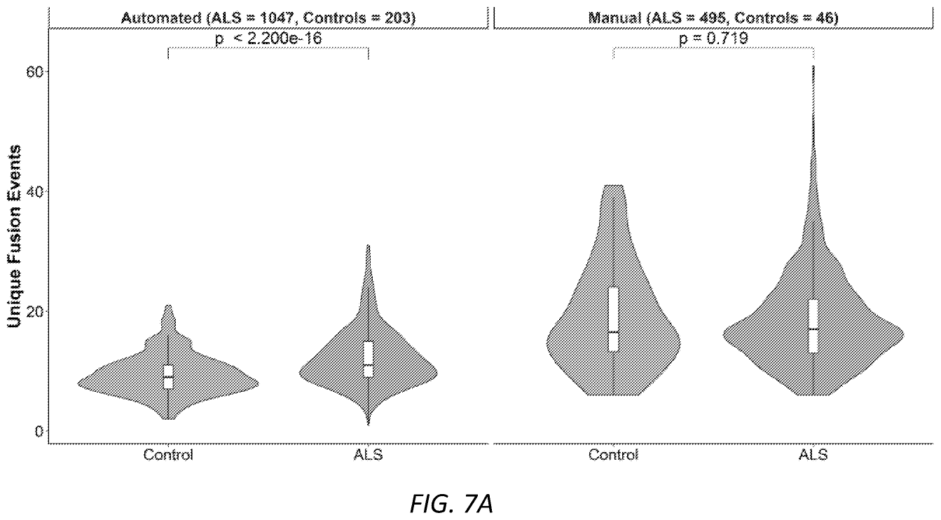

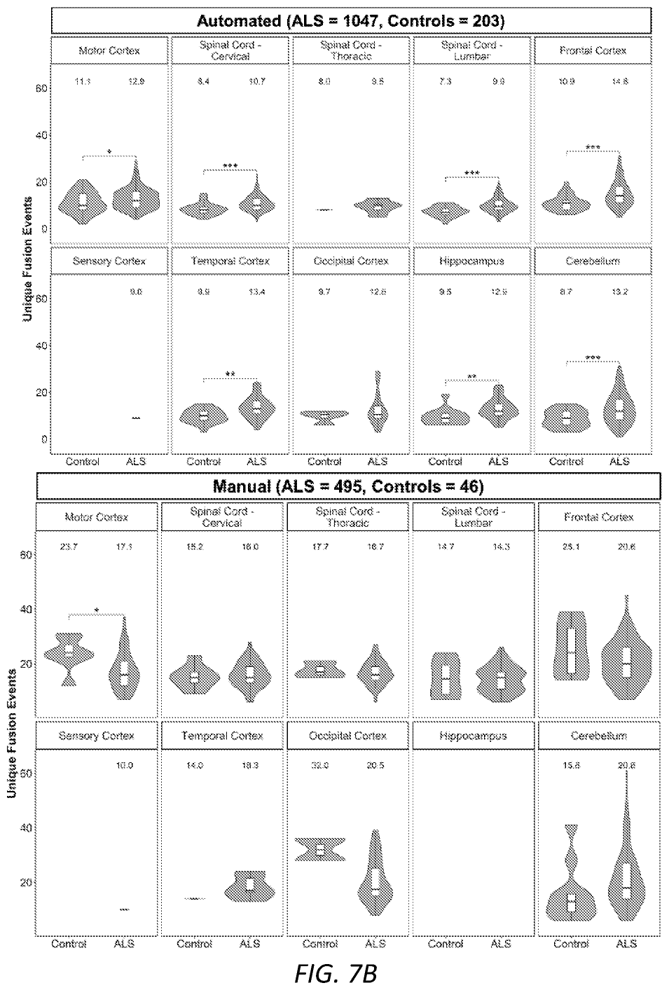

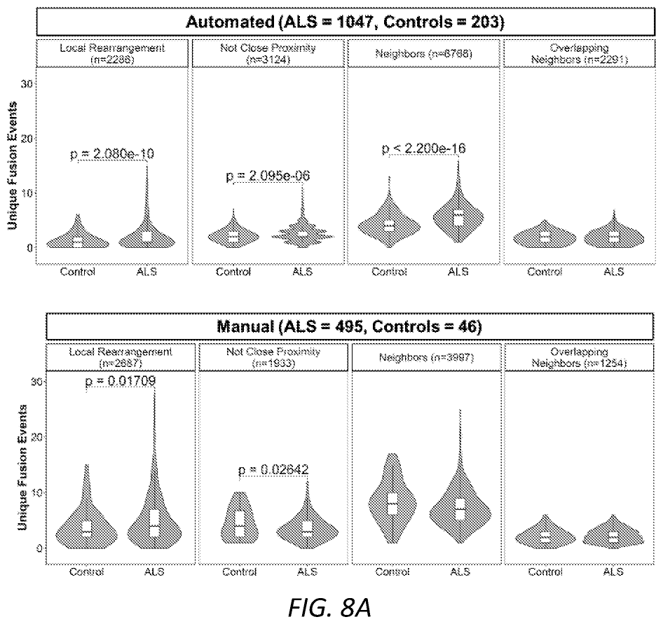

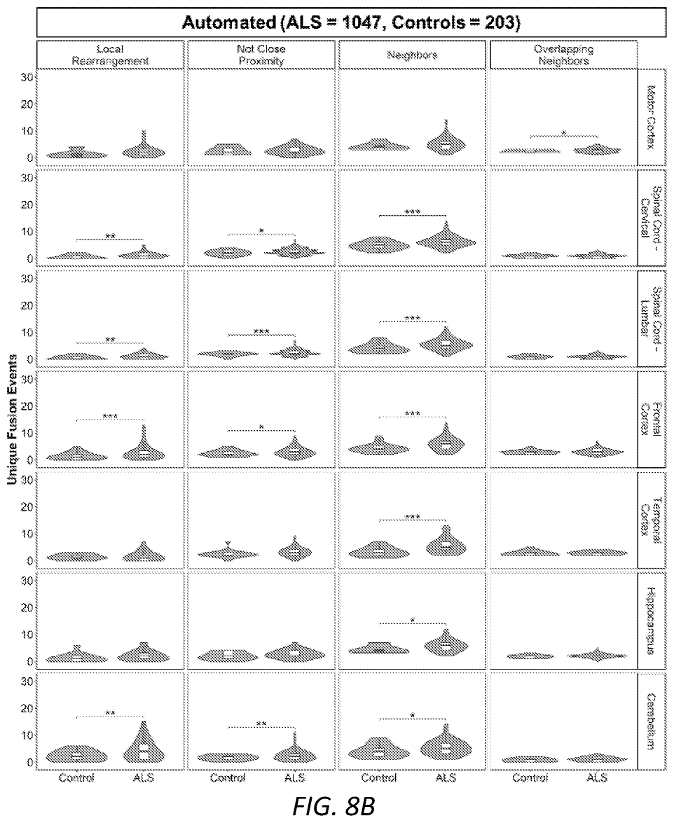

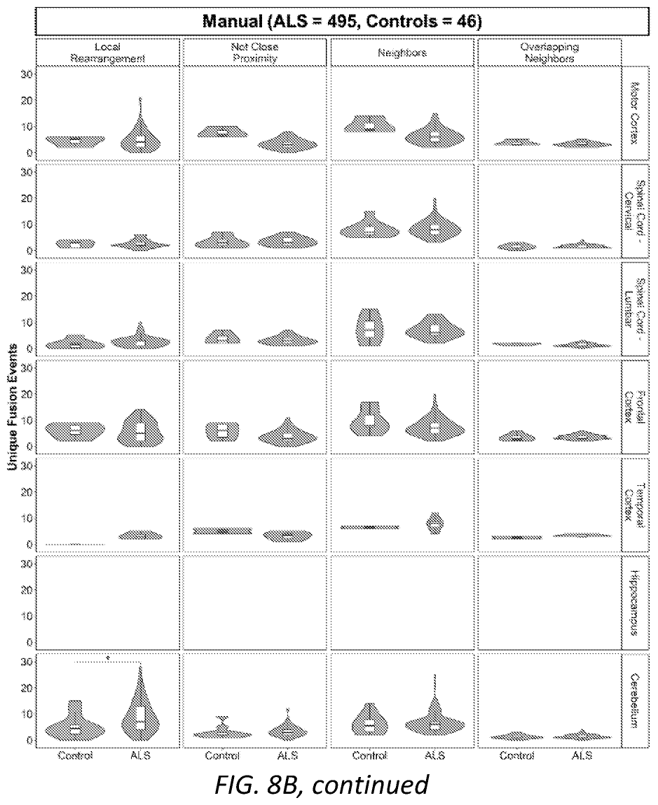

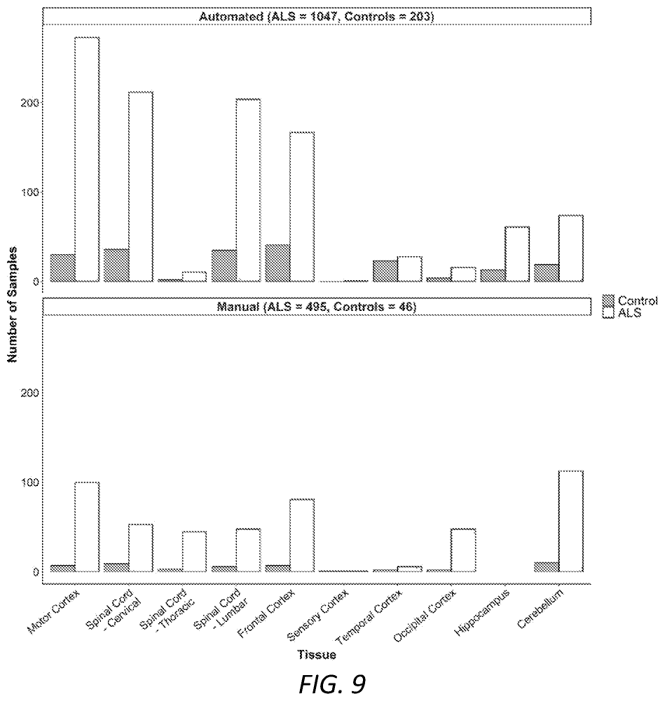

We also repeated all of the above analyses separately for samples that had gone through automated library capture preparation (1047 ALS and 203 controls) and manual library capture preparation, which was a much smaller group (495 ALS and 46 controls) (FIGS. 6A-C, 7A-B, 8A-B, 9, 10). The results for the subset with automated library preparation largely matched the findings presented above. Our analysis demonstrated that in the manual subset there was a significant enrichment of local rearrangement gene fusion events in all ALS tissues compared to controls (Welch's t-test, p=0.0171), and the significant enrichment of local rearrangements in ALS cerebellum compared to controls (Welch's t-test, p=0.0381) that captured the findings from the automated sample set. The remaining discrepancies in the results were likely due to the much smaller sample size and lack of controls in the manually prepared samples.

### Example 4. Individual Gene Fusion Burden

Next, we aimed to identify whether individual gene fusion pairs were driving the enrichment of gene fusions in ALS samples compared to controls. We identified specific gene fusion pairs with a significantly greater burden of breakpoint-unique gene fusions in ALS or control samples by applying the Fisher's exact test. Multiple testing corrected p-values were calculated using Bonferroni corrections based on the total number Fisher's exact tests and the F discovery rate method.

To determine whether any individual gene fusion pairs were driving the general enrichment of fusions in ALS in comparison to controls, burden analysis was applied to the full dataset. The top ten results from the gene fusion pair burden testing performed across all tissue samples are presented in Table 2; the top 90 significant ALS-Specific fusions are presented in Table 3 (none of the fusions in Table 3 appeared in any of the 249 control samples, and none appeared in a database). Importantly, these top ten included the only gene fusion pairs that displayed a significant burden following Bonferroni correction when comparing ALS to control samples across all tissues. To highlight gene fusions that may be unique to ALS samples, we also filtered the gene fusion burden results to only include rare gene fusion pairs, defined as those absent from known cancer databases and from the control samples (Table 4).

TABLE 2
Top 10 Gene fusion pairs with the highest individual burden in ALS versus control samples

| | | ALS | Control | | Lower | Upper | | Bonferroni | FDR |
|---|---|---|---|---|---|---|---|---|---|
| Fusion Name | Fusion Type | (n = 1542) | (n = 249) | OR | 95% CI | 95% CI | p-value | p-value | p-value |
| AC006427.2-- | Local | 471 | 34 | 2.78 | 1.89 | 4.19 | 8.76E−09 | 5.39E−06 | 3.48E−06 |
| TAPT1-AS1 | Rearrangement | | | | | | | | |
| KRTAP5-AS1-- | Not Close | 105 | 47 | 0.31 | 0.21 | 0.47 | 1.13E−08 | 6.95E−06 | 3.48E−06 |
| AP000867.5 | Proximity | | | | | | | | |
| DOCK4--IMMP2L | Not Close | 0 | 8 | 0.01 | 0.00 | 0.09 | 1.27E−07 | 7.78E−05 | 2.59E−05 |
| | Proximity | | | | | | | | |
| AC090517.5-- | Neighbors | 1116 | 140 | 2.04 | 1.53 | 2.71 | 5.02E−07 | 3.09E−04 | 7.72E−05 |
| ZNF280D | | | | | | | | | |
| TVP23C--CDRT4 | Neighbors | 940 | 110 | 1.97 | 1.49 | 2.61 | 7.70E−07 | 4.73E−04 | 9.47E−05 |
| AC009975.1-- | Local | 268 | 16 | 3.06 | 1.81 | 5.54 | 2.35E−06 | 1.44E−03 | 2.40E−04 |
| STON1-GTF2A1L | Rearrangement | | | | | | | | |
| MAILR-- | Neighbors | 301 | 20 | 2.78 | 1.72 | 4.71 | 3.02E−06 | 1.86E−03 | 2.65E−04 |
| ATP6V1C1 | | | | | | | | | |
| C8orf44--SGK3 | Neighbors | 202 | 11 | 3.26 | 1.75 | 6.74 | 2.06E−05 | 1.27E−02 | 1.59E−03 |
| PDC-AS1--ODR4 | Local | 222 | 15 | 2.62 | 1.52 | 4.85 | 1.18E−04 | 7.26E−02 | 8.07E−03 |
| | Rearrangement | | | | | | | | |
| FAM228A-- | Local | 59 | 0 | 20.01 | 1.21 | 318.39 | 1.85E−04 | 1.14E−01 | 1.14E−02 |
| FAM228B | Rearrangement | | | | | | | | |

Individual gene fusion burden tests were performed using the Fisher's exact test. Bonferroni and FDR corrections were based on the total number of fusions across all tissues (n = 607).
Abbreviations: ALS, amyotrophic lateral sclerosis; CI, confidence interval; FDR, F discovery rate; OR, odds ratio.

TABLE 3
Top 90 Significant Gene fusions in ALS versus control samples

| | | | | | | | | | | Fisher's |
|---|---|---|---|---|---|---|---|---|---|---|
| | | | | | | | | | Fisher's | Exact |
| | Cases | Cases | Inter vs | | Intra | | Fisher's | Fisher's | Exact FDR- | Bonferroni- |
| | with | without | Intrachro- | Close | Fusion | | Exact Odds | Exact Raw | Adjusted | Adjusted |
| Index_Col | Fusion | Fusion | mosomal | Proximity | Type | Databases | Ratio | P-Value | P-Value | P-Value |
| FAM228A-- | 59 | 1483 | Intra | T | | None | inf | 0.000185 | 0.011384 | 0.113842 |
| FAM228B | | | | | | | | | | |
| YAF2--RYBP | 33 | 1509 | Inter | F | N/A | None | inf | 0.010256 | 0.191139 | 1 |
| AC108047.1-- | 20 | 1522 | Intra | T | Nb | None | inf | 0.09747 | 0.855025 | 1 |
| NAB1 | | | | | | | | | | |
| RMND5A-- | 19 | 1523 | Intra | F | NCP | None | inf | 0.09533 | 0.855025 | 1 |
| ANAPC1P2 | | | | | | | | | | |
| AC009975.1-- | 19 | 1523 | Intra | T | LR | None | inf | 0.09533 | 0.855025 | 1 |
| GTF2A1L | | | | | | | | | | |
| LINC01736-- | 19 | 1523 | Intra | T | Nb | None | inf | 0.09533 | 0.855025 | 1 |
| GALNT2 | | | | | | | | | | |
| AC037433.1-- | 19 | 1523 | Intra | T | Nb | None | inf | 0.09533 | 0.855025 | 1 |
| AC009654.1 | | | | | | | | | | |
| AC092747.2-- | 16 | 1526 | Intra | T | LR | None | inf | 0.149611 | 0.868027 | 1 |
| MED21 | | | | | | | | | | |
| AC097500.1-- | 16 | 1526 | Intra | F | NCP | None | inf | 0.149611 | 0.868027 | 1 |
| FSIP2-AS1 | | | | | | | | | | |
| GTF2IP9-- | 15 | 1527 | Intra | T | LR | None | inf | 0.248931 | 1 | 1 |
| AC008267.2 | | | | | | | | | | |
| LRRC37A--NSF | 14 | 1528 | Intra | F | NCP | None | inf | 0.239847 | 1 | 1 |
| AC073529.1-- | 11 | 1531 | Intra | T | Nb | None | inf | 0.37991 | 1 | 1 |
| MID1 | | | | | | | | | | |
| AC003044.1-- | 11 | 1531 | Intra | T | Nb | None | inf | 0.37991 | 1 | 1 |
| AC004485.1 | | | | | | | | | | |
| AC096642.1-- | 11 | 1531 | Intra | T | LR | None | inf | 0.37991 | 1 | 1 |
| LYPLAL1 | | | | | | | | | | |
| SHPRH--FBXO30 | 10 | 1532 | Intra | T | Nb | None | inf | 0.374544 | 1 | 1 |
| AC135983.3-- | 10 | 1532 | Intra | T | LR | None | inf | 0.374544 | 1 | 1 |
| AC135983.2 | | | | | | | | | | |
| AC126182.3-- | 10 | 1532 | Intra | T | Nb | None | inf | 0.374544 | 1 | 1 |
| NDUFB4 | | | | | | | | | | |
| AC021851.1-- | 9 | 1533 | Intra | T | LR | None | inf | 0.621469 | 1 | 1 |
| NUP35 | | | | | | | | | | |
| CFAP299--FGF5 | 9 | 1533 | Intra | T | NbO | None | inf | 0.621469 | 1 | 1 |
| MIA2-- | 8 | 1534 | Intra | T | Nb | None | inf | 0.609167 | 1 | 1 |
| AL049828.1 | | | | | | | | | | |
| SDHAP3-- | 8 | 1534 | Intra | T | LR | None | inf | 0.609167 | 1 | 1 |
| AC026412.1 | | | | | | | | | | |
| FBXO25-- | 8 | 1534 | Inter | F | N/A | None | inf | 0.609167 | 1 | 1 |
| SEPTIN14 | | | | | | | | | | |
| PICALM--SYTL2 | 7 | 1535 | Intra | F | NCP | None | inf | 0.602805 | 1 | 1 |
| APP-DT-- | 7 | 1535 | Intra | F | NCP | None | inf | 0.602805 | 1 | 1 |
| AP001596.1 | | | | | | | | | | |
| AC007221.1-- | 7 | 1535 | Intra | F | NCP | None | inf | 0.602805 | 1 | 1 |
| AC007218.2 | | | | | | | | | | |
| AGAP5-- | 7 | 1535 | Intra | T | LR | None | inf | 0.602805 | 1 | 1 |
| AC022400.4 | | | | | | | | | | |
| AC006299.1-- | 7 | 1535 | Intra | F | NCP | None | inf | 0.602805 | 1 | 1 |
| LINC02718 | | | | | | | | | | |
| RPS25P9--PPA1 | 7 | 1535 | Intra | T | LR | None | inf | 0.602805 | 1 | 1 |
| ZBTB38--RASA2 | 7 | 1535 | Intra | T | Nb | None | inf | 0.602805 | 1 | 1 |
| BNIP3L--DPYSL2 | 7 | 1535 | Intra | T | Nb | None | inf | 0.602805 | 1 | 1 |
| CFAP299--BMP3 | 7 | 1535 | Intra | T | Nb | None | inf | 0.602805 | 1 | 1 |
| FSIP2-AS1-- | 7 | 1535 | Intra | F | NCP | None | inf | 0.602805 | 1 | 1 |
| AC097500.1 | | | | | | | | | | |
| AC138627.1-- | 6 | 1536 | Intra | T | Nb | None | inf | 1 | 1 | 1 |
| ZFHX3 | | | | | | | | | | |
| SLC20A2--IDO2 | 6 | 1536 | Intra | F | NCP | None | inf | 1 | 1 | 1 |
| ST8SIA3-- | 6 | 1536 | Intra | T | Nb | None | inf | 1 | 1 | 1 |
| ONECUT2 | | | | | | | | | | |
| AC104596.1-- | 6 | 1536 | Intra | T | Nb | None | inf | 1 | 1 | 1 |
| INPP4B | | | | | | | | | | |
| NFIA-AS2-- | 5 | 1537 | Intra | T | Nb | None | inf | 1 | 1 | 1 |
| AC099792.1 | | | | | | | | | | |
| SH3GL2-- | 5 | 1537 | Intra | F | NCP | None | inf | 1 | 1 | 1 |
| ADAMTSL1 | | | | | | | | | | |
| LINC00674-- | 5 | 1537 | Intra | T | Nb | None | inf | 1 | 1 | 1 |
| AMZ2 | | | | | | | | | | |
| AC079382.2-- | 5 | 1537 | Intra | T | LR | None | inf | 1 | 1 | 1 |
| LINC00882 | | | | | | | | | | |
| RTN1--CCDC175 | 5 | 1537 | Intra | T | Nb | None | inf | 1 | 1 | 1 |
| AC110751.1-- | 5 | 1537 | Intra | T | LR | None | inf | 1 | 1 | 1 |
| AC105383.1 | | | | | | | | | | |
| ANGEL1-- | 5 | 1537 | Intra | T | Nb | None | inf | 1 | 1 | 1 |
| AC008050.1 | | | | | | | | | | |
| AL121890.4-- | 5 | 1537 | Intra | T | LR | None | inf | 1 | 1 | 1 |
| TMEM230 | | | | | | | | | | |
| KANSL1L--ACADL | 5 | 1537 | Intra | T | LR | None | inf | 1 | 1 | 1 |
| LRRC37A2--NSF | 5 | 1537 | Intra | T | Nb | None | inf | 1 | 1 | 1 |
| SORL1-- | 5 | 1537 | Intra | T | Nb | None | inf | 1 | 1 | 1 |
| AP001977.1 | | | | | | | | | | |
| NAIP--BDP1 | 5 | 1537 | Intra | F | NCP | None | inf | 1 | 1 | 1 |
| CPLANE1-- | 5 | 1537 | Intra | T | LR | None | inf | 1 | 1 | 1 |
| NUP155 | | | | | | | | | | |
| AC093843.1-- | 5 | 1537 | Intra | T | LR | None | inf | 1 | 1 | 1 |
| AC067956.1 | | | | | | | | | | |
| TARS3--OR4F17 | 5 | 1537 | Inter | F | N/A | None | inf | 1 | 1 | 1 |
| AC245297.3-- | 5 | 1537 | Intra | F | NCP | None | inf | 1 | 1 | 1 |
| SEC22B | | | | | | | | | | |
| AC004828.2-- | 5 | 1537 | Intra | T | LR | None | inf | 1 | 1 | 1 |
| AC004828.1 | | | | | | | | | | |
| LHFPL5--CLPSL1 | 4 | 1538 | Intra | T | LR | None | inf | 1 | 1 | 1 |
| CCDC62--DENR | 4 | 1538 | Intra | T | LR | None | inf | 1 | 1 | 1 |
| AP001347.1-- | 4 | 1538 | Intra | T | Nb | None | inf | 1 | 1 | 1 |
| RBM11 | | | | | | | | | | |
| PABPC1--SNX31 | 4 | 1538 | Intra | T | Nb | None | inf | 1 | 1 | 1 |
| PHOSPHO2--SSB | 4 | 1538 | Intra | T | Nb | None | inf | 1 | 1 | 1 |
| PRELID2-- | 4 | 1538 | Intra | T | Nb | None | inf | 1 | 1 | 1 |
| AC137770.1 | | | | | | | | | | |
| AC114971.1-- | 4 | 1538 | Intra | F | NCP | None | inf | 1 | 1 | 1 |
| LINC02144 | | | | | | | | | | |
| SMIM10L1-- | 4 | 1538 | Intra | F | NCP | None | inf | 1 | 1 | 1 |
| LINC01252 | | | | | | | | | | |
| AC138393.1-- | 4 | 1538 | Inter | F | N/A | None | inf | 1 | 1 | 1 |
| SEPTIN14 | | | | | | | | | | |
| FAM106A-- | 4 | 1538 | Intra | T | NbO | None | inf | 1 | 1 | 1 |
| CCDC144B | | | | | | | | | | |
| AL365259.1-- | 4 | 1538 | Intra | F | NCP | None | inf | 1 | 1 | 1 |
| AL450332.1 | | | | | | | | | | |
| LINC02296-- | 4 | 1538 | Intra | T | LR | None | inf | 1 | 1 | 1 |
| LINC02330 | | | | | | | | | | |
| AC010332.3-- | 4 | 1538 | Intra | T | LR | None | inf | 1 | 1 | 1 |
| ZNF534 | | | | | | | | | | |
| AL109809.2-- | 4 | 1538 | Intra | T | LR | None | inf | 1 | 1 | 1 |
| AL109809.4 | | | | | | | | | | |
| MALAT1--GFAP | 4 | 1538 | Inter | F | N/A | None | inf | 1 | 1 | 1 |
| AC009093.8-- | 4 | 1538 | Intra | T | LR | None | inf | 1 | 1 | 1 |
| AC009093.9 | | | | | | | | | | |
| AC240565.1-- | 3 | 1539 | Inter | F | N/A | None | inf | 1 | 1 | 1 |
| AP006222.1 | | | | | | | | | | |
| AC103874.1-- | 3 | 1539 | Intra | F | NCP | None | inf | 1 | 1 | 1 |
| NEO1 | | | | | | | | | | |
| KTN1-- | 3 | 1539 | Intra | F | NCP | None | inf | 1 | 1 | 1 |
| AL163952.1 | | | | | | | | | | |
| AC016907.2-- | 3 | 1539 | Intra | T | Nb | None | inf | 1 | 1 | 1 |
| AC106870.3 | | | | | | | | | | |
| AP002364.1-- | 3 | 1539 | Intra | T | Nb | None | inf | 1 | 1 | 1 |
| DISC1FP1 | | | | | | | | | | |
| LINC02821-- | 3 | 1539 | Intra | T | Nb | None | inf | 1 | 1 | 1 |
| CNOT2 | | | | | | | | | | |
| AC104653.2-- | 3 | 1539 | Intra | T | NbO | None | inf | 1 | 1 | 1 |
| AC104653.1 | | | | | | | | | | |
| LINC01138-- | 3 | 1539 | Intra | F | NCP | None | inf | 1 | 1 | 1 |
| SEC22B | | | | | | | | | | |
| ANKAR--ASNSD1 | 3 | 1539 | Intra | T | LR | None | inf | 1 | 1 | 1 |
| AP001372.2-- | 3 | 1539 | Intra | T | Nb | None | inf | 1 | 1 | 1 |
| POLD3 | | | | | | | | | | |
| ERP44-- | 3 | 1539 | Intra | T | Nb | None | inf | 1 | 1 | 1 |
| STX17-AS1 | | | | | | | | | | |
| AL669831.1-- | 3 | 1539 | Inter | F | N/A | None | inf | 1 | 1 | 1 |
| SEPTIN14 | | | | | | | | | | |
| MED6-- | 3 | 1539 | Intra | T | Nb | None | inf | 1 | 1 | 1 |
| AL357153.2 | | | | | | | | | | |
| SLC5A3-- | 3 | 1539 | Intra | T | Nb | None | inf | 1 | 1 | 1 |
| AP000317.1 | | | | | | | | | | |
| AL163540.2-- | 3 | 1539 | Intra | T | LR | None | inf | 1 | 1 | 1 |
| FLJ43315 | | | | | | | | | | |
| AC123767.1-- | 3 | 1539 | Intra | F | NCP | None | inf | 1 | 1 | 1 |
| ADAM5 | | | | | | | | | | |
| CFAP69-- | 3 | 1539 | Intra | F | NCP | None | inf | 1 | 1 | 1 |
| AC002069.2 | | | | | | | | | | |
| NKAPP1-- | 3 | 1539 | Intra | T | Nb | None | inf | 1 | 1 | 1 |
| LINC01402 | | | | | | | | | | |
| RNF138-- | 3 | 1539 | Intra | T | Nb | None | inf | 1 | 1 | 1 |
| MEP1B | | | | | | | | | | |
| AL669831.1-- | 3 | 1539 | Intra | F | NCP | None | inf | 1 | 1 | 1 |
| AP006222.1 | | | | | | | | | | |
| FAM157B-- | 3 | 1539 | Inter | F | N/A | None | inf | 1 | 1 | 1 |
| AP006222.1 | | | | | | | | | | |

T, True;
F, False;
LR, Local Rearrangement;
N/A, Not Applicable;
NCP, Not Close Proximity;
Nb, Neighbors;
NbO, Neighbors Overlap

TABLE 4
Rare gene fusion pairs with the highest individual burden in ALS versus control samples.

| | | ALS | Control | | Lower | Upper | | Bonferroni | FDR |
|---|---|---|---|---|---|---|---|---|---|
| Fusion Name | Fusion Type | (n = 1542) | (n = 249) | OR | 95% CI | 95% CI | p-value | p-value | p-value |
| YAF2--RYBP | Inter- | 33 | 0 | 11.07 | 1.38 | Inf | 1.03E−02 | 1.00 | 1.00 |
| | chromosomal | | | | | | | | |
| FAM228A-- | Local | 59 | 0 | 20.01 | 2.58 | Inf | 1.85E−04 | 5.26E−02 | 5.26E−02 |
| FAM228B | Rearrangement | | | | | | | | |
| AC009975.1-- | Local | 19 | 0 | 6.39 | 0.76 | Inf | 9.53E−02 | 1.00 | 1.00 |
| GTF2A1L | Rearrangement | | | | | | | | |
| AC037433.1-- | Neighbors | 19 | 0 | 6.39 | 0.76 | Inf | 9.53E−02 | 1.00 | 1.00 |
| AC009654.1 | | | | | | | | | |
| LINC01736-- | Neighbors | 19 | 0 | 6.39 | 0.76 | Inf | 9.53E−02 | 1.00 | 1.00 |
| GALNT2 | | | | | | | | | |
| RMND5A-- | Not Close | 19 | 0 | 6.39 | 0.76 | Inf | 9.53E−02 | 1.00 | 1.00 |
| ANAPC1P2 | Proximity | | | | | | | | |
| AC108047.1-- | Neighbors | 20 | 0 | 6.72 | 0.80 | Inf | 9.75E−02 | 1.00 | 1.00 |
| NAB1 | | | | | | | | | |
| AC092747.2-- | Local | 16 | 0 | 5.39 | 0.62 | Inf | 1.50E−01 | 1.00 | 1.00 |
| MED21 | Rearrangement | | | | | | | | |
| AC097500.1-- | Not Close | 16 | 0 | 5.39 | 0.62 | Inf | 1.50E−01 | 1.00 | 1.00 |
| FSIP2-AS1 | Proximity | | | | | | | | |
| LRRC37A--NSF | Not Close | 14 | 0 | 4.73 | 0.54 | Inf | 2.40E−01 | 1.00 | 1.00 |
| | Proximity | | | | | | | | |

Individual gene fusion burden tests were performed using the Fisher's exact test and results were prioritized to identify only include rare breakpoint-unique gene fusions, which were defined as those absent from known fusion databases and absent from the control samples. Bonferroni and FDR corrections were based on the total number of rare fusions across all tissues (n = 280).
Abbreviations: ALS, amyotrophic lateral sclerosis; CI, confidence interval; FDR, F discovery rate; OR, odds ratio.

Based on the results presented in FIGS. 4A-B, we next aimed to determine whether specific gene fusions were driving enrichment of specific intra-chromosomal subtypes. To maximize statistical power and minimize potential signal from tissues not displaying enrichment of gene fusion pairs, we binned together all gene fusion pairs carried by samples from tissues displaying significant enrichments of fusion of that specific subtype. Therefore, samples from the following tissue sources were binned: motor cortex, cervical spinal cord, lumbar spinal cord, frontal cortex, and cerebellum in the burden test of local rearrangement fusions; cerebellum in the burden test of not close proximity fusions; cervical spinal cord, lumbar spinal cord, frontal cortex, temporal cortex, hippocampus, and cerebellum in the burden test of neighbor fusions; and motor cortex and frontal cortex in the burden test of overlapping neighbor fusions. We then performed an individual gene burden test for each intra-chromosomal gene fusion subtype using these binned groups of tissue sources (Table 5). Again, the gene fusion burden results were filtered to only include rare gene fusion pairs, defined as those absent from known cancer databases and from control samples (Table 6).

TABLE 5
Intra-chromosomal gene fusion pairs identified in tissues displaying significant enrichment
in ALS samples with the highest individual burden in ALS versus controls

Intra-

| chromosomal | | ALS | | Control | | | Lower | Upper | | Bonferroni | FDR |
|---|---|---|---|---|---|---|---|---|---|---|---|
| Fusion Type | Fusion Name | (n) | | (n) | | OR | 95% CI | 95% CI | p-value | p-value | p-value |
| Local | AC006427.2-- | 402 | (1325) | 26 | (200) | 2.91 | 1.89 | 4.66 | 7.95E−08 | 1.11E−05 | 1.11E−05 |
| Rearrangement | TAPT1-AS1 | | | | | | | | | | |
| | AC009975.1-- | 234 | (1325) | 16 | (200) | 2.47 | 1.44 | 4.49 | 3.09E−04 | 4.33E−02 | 2.16E−02 |
| | STON1-GTF2A1L | | | | | | | | | | |
| | ADAMTSL3-- | 281 | (1325) | 22 | (200) | 2.18 | 1.36 | 3.63 | 5.72E−04 | 8.01E−02 | 2.53E−02 |
| | SH3GL3 | | | | | | | | | | |
| Not Close | ABCD2--KIF21A | 67 | (187) | 2 | (29) | 7.49 | 1.79 | 67.06 | 1.12E−03 | 6.51E−02 | 9.79E−01 |
| Proximity | PMS2P11-- | 0 | (187) | 2 | (29) | 0.03 | 0.00 | 0.81 | 1.75E−02 | 1.00 | 9.79E−01 |
| | CCDC146 | | | | | | | | | | |
| | AC023421.2-- | 0 | (187) | 1 | (29) | 0.05 | 0.00 | 6.05 | 1.34E−01 | 1.00 | 1.00 |
| | AC021517.2 | | | | | | | | | | |
| Neighbor | TVP23C--CDRT4 | 633 | (1047) | 82 | (201) | 2.22 | 1.61 | 3.06 | 3.48E−07 | 6.74E−05 | 6.74E−05 |
| | C8orf44--SGK3 | 159 | (1047) | 8 | (201) | 4.32 | 2.09 | 10.35 | 2.62E−06 | 5.08E−04 | 2.54E−04 |
| | AC090517.5-- | 728 | (1047) | 109 | (201) | 1.93 | 1.40 | 2.65 | 3.84E−05 | 7.44E−03 | 2.48E−03 |
| | ZNF280D | | | | | | | | | | |
| Overlapping | AC127502.3-- | 91 | (621) | 4 | (85) | 3.47 | 1.26 | 13.37 | 1.02E−02 | 2.54E−01 | 2.54E−01 |
| Neighbor | AC127502.1 | | | | | | | | | | |
| | AC067956.1-- | 131 | (621) | 9 | (85) | 2.26 | 1.09 | 5.26 | 2.05E−02 | 5.13E−01 | 2.56E−01 |
| | AC019211.1 | | | | | | | | | | |
| | CLDN12--CDK14 | 42 | (621) | 3 | (85) | 1.98 | 0.61 | 10.22 | 3.45E−01 | 1.00 | 1.00 |

The individual burden tests of gene fusion events were only performed on fusions within tissues demonstrating significant differences in the number of gene fusions of each intra-chromosomal subtype carried by ALS and control patients. Only samples from tissues showing significant enrichment of that specific subtype were included in each burden test, including samples from the motor cortex, cervical spinal cord, lumbar spinal cord, frontal cortex, and cerebellum in the burden test of local rearrangement fusions; cerebellum in the burden test of not close proximity fusions; cervical spinal cord, lumbar spinal cord, frontal cortex, temporal cortex, hippocampus, and cerebellum in the burden test of neighbor fusions; and motor cortex and frontal cortex in the burden test of overlapping neighbor fusions. Bonferroni and FDR corrections were based on the total number of fusions observed within the respective tissues.
Abbreviations:
ALS, amyotrophic lateral sclerosis;
CI, confidence interval;
FDR, F discovery rate;
n, total number of samples;
OR, odds ratio

TABLE 6
Rare intra-chromosomal gene fusion pairs identified in tissues displaying significant
enrichment in ALS samples with the highest individual burden in ALS versus controls.

Intra-

| chromosomal | | ALS | | Control | | | Lower | Upper | | Bonferroni | FDR |
|---|---|---|---|---|---|---|---|---|---|---|---|
| Fusion Type | Fusion Name | (n) | | (n) | | OR | 95% CI | 95% CI | p-value | p-value | p-value |
| Local | FAM228A-- | 53 | (1325) | 0 | (200) | 16.86 | 2.16 | Inf | 7.23E−04 | 4.77E−02 | 4.77E−02 |
| Rearrangement | FAM228B | | | | | | | | | | |
| | AC092747.2-- | 15 | (1325) | 0 | (200) | 4.74 | 0.54 | Inf | 2.43E−01 | 1.00 | 1.00 |
| | MED21 | | | | | | | | | | |
| | AC110023.1-- | 15 | (1325) | 0 | (200) | 4.74 | 0.54 | Inf | 2.43E−01 | 1.00 | 1.00 |
| | AC091078.1 | | | | | | | | | | |
| Not Close | RMND5A-- | 16 | (187) | 0 | (29) | 5.68 | 0.61 | Inf | 1.37E−01 | 1.00 | 1.00 |
| Proximity | ANAPC1P2 | | | | | | | | | | |
| | BMS1P7--PTPN20 | 10 | (187) | 0 | (29) | 3.49 | 0.35 | Inf | 3.65E−01 | 1.00 | 1.00 |
| | TRMT11--CENPW | 10 | (187) | 0 | (29) | 3.49 | 0.35 | Inf | 3.65E−01 | 1.00 | 1.00 |
| Neighbor | SLC25A32-- | 24 | (1047) | 0 | (201) | 9.45 | 1.17 | Inf | 2.29E−02 | 1.00 | 1.00 |
| | BAALC-AS1 | | | | | | | | | | |
| | AC037433.1-- | 17 | (1047) | 0 | (201) | 6.84 | 0.80 | Inf | 9.18E−02 | 1.00 | 1.00 |
| | AC009654.1 | | | | | | | | | | |
| | AC108047.1-- | 11 | (1047) | 0 | (201) | 4.47 | 0.48 | Inf | 2.29E−01 | 1.00 | 1.00 |
| | NAB1 | | | | | | | | | | |
| Overlapping | CLDN11-- | 10 | (621) | 0 | (85) | 2.94 | 0.31 | Inf | 6.18E−01 | 1.00 | 1.00 |
| Neighbor | SLC7A14-AS1 | | | | | | | | | | |
| | AC233976.1-- | 6 | (621) | 0 | (85) | 1.81 | 0.16 | Inf | 1.00 | 1.00 | 1.00 |
| | LINC01284 | | | | | | | | | | |
| | TIMM23B- | 3 | (621) | 0 | (85) | 0.97 | 0.06 | Inf | 1.00 | 1.00 | 1.00 |
| | AGAP6-- | | | | | | | | | | |
| | TIMM23B | | | | | | | | | | |

The individual burden tests of gene fusion events were only performed on fusions within tissues demonstrating significant differences in the number of gene fusions of each intra-chromosomal subtype carried by ALS and control patients. Only samples from tissues showing significant enrichment of that specific subtype were included in each burden test, including samples from the motor cortex, cervical spinal cord, lumbar spinal cord, frontal cortex, and cerebellum in the burden test of local rearrangement fusions; cerebellum in the burden test of not close proximity fusions; cervical spinal cord, lumbar spinal cord, frontal cortex, temporal cortex, hippocampus, and cerebellum in the burden test of neighbor fusions; and motor cortex and frontal cortex in the burden test of overlapping neighbor fusions. Results were prioritized to only include rare breakpoint-unique gene fusions, which were defined as those absent from known fusion databases and absent from the control samples. Bonferroni and FDR corrections were based on the total number of fusions observed within the respective tissues.
Abbreviations: ALS, amyotrophic lateral sclerosis; CI, confidence interval; FDR, F discovery rate; n, total number of samples; OR, odds ratio.

Finally, burden testing was performed for each intra-chromosomal subtype for gene fusion pairs identified in each tissue displaying significant enrichment of fusion of that specific subtype (Table 7), to determine if individual gene fusions were driving the signals of enrichment in the tissue. Following multiple testing correction, significant burdens in ALS samples compared to controls were found for one local rearrangement in both the cervical spinal cord samples (AC006427.2-TAPT1-AS1; OR=3.70 [1.62-9.55]; Fisher's test, p=0.0404, following multiple testing correction) and lumbar spinal cord samples (AC006427.2-TAPT1-AS1; OR=5.96 [2.05-40.92]; Fisher's test, p=0.0075, following multiple testing correction), one neighbor fusion in the cervical spinal cord samples (PAMR1-SLC1A2; OR=24.54 [3.00-Inf]; Fisher's test, p=0.0085, following multiple testing correction), one neighbor fusion in the temporal cortex samples (AEBP2-AC024901.1; OR=32.02 [3.08-Inf]; Fisher's test, p=0.0112, following multiple testing correction), and one overlapping neighbor fusion in the frontal cortex samples (AC067956.1-AC019211.1; OR=4.40 [1.52-17.48]; Fisher's test, p=0.0432, following multiple testing correction).

TABLE 7
Top intra-chromosomal gene fusion pairs in each tissue displaying significant enrichment in ALS samples versus controls.

| | | | | | | | | | |
|---|---|---|---|---|---|---|---|---|---|
| | | ALS | Control | | Lower | Upper | | Bonferroni | FDR |
| Tissue Type | Fusion Name | (n) | (n) | OR | 95% CI | 95% CI | p-value | p-value | p-value |

Intra-chromosomal Fusion Type: Local Rearrangement

| | | | | | | | | | | | |
|---|---|---|---|---|---|---|---|---|---|---|---|
| Motor Cortex | CAVIN4--TMEFF1 | 201 | (373) | 12 | (37) | 2.43 | 1.14 | 5.48 | 1.53E−02 | 1.00 | 1.00 |
| | AL731769.2-- | 0 | (373) | 1 | (37) | 0.03 | 0.00 | 3.87 | 9.02E−02 | 1.00 | 1.00 |
| | SCART1 | | | | | | | | | | |
| | ERMP1-- | 28 | (373) | 0 | (37) | 6.19 | 0.72 | Inf | 9.49E−02 | 1.00 | 1.00 |
| | KIAA2026 | | | | | | | | | | |
| Spinal Cord - | AC006427.2-- | 118 | (265) | 8 | (45) | 3.70 | 1.62 | 9.55 | 8.59E−04 | 4.04E−02 | 4.04E−02 |
| Cervical | TAPT1-AS1 | | | | | | | | | | |
| | AF241728.1-- | 0 | (265) | 2 | (45) | 0.03 | 0.00 | 0.89 | 2.07E−02 | 9.72E−01 | 4.86E−01 |
| | TSPAN7 | | | | | | | | | | |
| | CLIC2-- | 3 | (265) | 3 | (45) | 0.16 | 0.02 | 1.25 | 4.18E−02 | 1.00 | 6.55E−01 |
| | AC234781.1 | | | | | | | | | | |
| Spinal Cord - | AC006427.2-- | 99 | (252) | 4 | (41) | 5.96 | 2.05 | 23.72 | 1.50E−04 | 7.48E−03 | 7.48E−03 |
| Lumbar | TAPT1-AS1 | | | | | | | | | | |
| | AC009975.1-- | 85 | (252) | 5 | (41) | 3.65 | 1.36 | 12.36 | 5.61E−03 | 2.80E−01 | 1.40E−01 |
| | STON1-GTF2A1L | | | | | | | | | | |
| | AC012414.4-- | 0 | (252) | 1 | (41) | 0.05 | 0.00 | 6.35 | 1.40E−01 | 1.00 | 1.00 |
| | AC012414.2 | | | | | | | | | | |
| | RANBP2--LIMS1 | 0 | (252) | 1 | (41) | 0.05 | 0.00 | 6.35 | 1.40E−01 | 1.00 | 1.00 |
| | SMG8--PRR11 | 0 | (252) | 1 | (41) | 0.05 | 0.00 | 6.35 | 1.40E−01 | 1.00 | 1.00 |
| Frontal Cortex | AC006427.2-- | 89 | (248) | 6 | (48) | 3.90 | 1.57 | 11.68 | 1.18E−03 | 9.07E−02 | 5.45E−02 |
| | TAPT1-AS1 | | | | | | | | | | |
| | AC096642.2-- | 38 | (248) | 0 | (48) | 17.74 | 2.14 | Inf | 1.45E−03 | 1.12E−01 | 5.45E−02 |
| | LYPLAL1 | | | | | | | | | | |
| | CAVIN4--TMEFF1 | 111 | (248) | 10 | (48) | 3.07 | 1.42 | 7.22 | 2.12E−03 | 1.63E−01 | 5.45E−02 |
| Cerebellum | L3HYPDH-- | 107 | (187) | 7 | (29) | 4.18 | 1.62 | 12.16 | 1.14E−03 | 9.84E−02 | 6.85E−02 |
| | CCDC175 | | | | | | | | | | |
| | KPNA2P3-- | 89 | (187) | 5 | (29) | 4.33 | 1.53 | 15.17 | 2.22E−03 | 1.91E−01 | 6.85E−02 |
| | BPTFP1 | | | | | | | | | | |
| | AC009975.1-- | 52 | (187) | 1 | (29) | 10.72 | 1.68 | 448.50 | 2.39E−03 | 2.05E−01 | 6.85E−02 |
| | STON1-GTF2A1L | | | | | | | | | | |

Intra-chromosomal Fusion Type: Not Close Proximity

| | | | | | | | | | | | |
|---|---|---|---|---|---|---|---|---|---|---|---|
| Cerebellum | ABCD2--KIF21A | 66 | (182) | 2 | (28) | 7.35 | 1.75 | 65.94 | 1.85E−03 | 6.19E−02 | 6.19E−02 |
| | PMS2P11-- | 0 | (182) | 2 | (28) | 0.03 | 0 | 0.80 | 1.72E−02 | 9.99E−01 | 5.00E−01 |
| | CCDC146 | | | | | | | | | | |
| | AC023421.2-- | 0 | (182) | 1 | (28) | 0.05 | 0 | 6.00 | 1.33E−01 | 1.00 | 1.00 |
| | AC021517.2 | | | | | | | | | | |
| | DOCK4--IMMP2L | 0 | (182) | 1 | (28) | 0.05 | 0 | 6.00 | 1.33E−01 | 1.00 | 1.00 |
| | PCSK6--TARS3 | 0 | (182) | 1 | (28) | 0.05 | 0 | 6.00 | 1.33E−01 | 1.00 | 1.00 |

Intra-chromosomal Fusion Type: Neighbor

| | | | | | | | | | | | |
|---|---|---|---|---|---|---|---|---|---|---|---|
| Spinal Cord - | PAMR1--SLC1A2 | 56 | (265) | 0 | (45) | 24.54 | 3.00 | Inf | 8.94E−05 | 8.50E−03 | 8.50E−03 |
| Cervical | | | | | | | | | | | |
| | C8orf44--SGK3 | 42 | (265) | 1 | (45) | 8.26 | 1.33 | 342.05 | 9.86E−03 | 9.37E−01 | 4.51E−01 |
| | MAILR-- | 60 | (265) | 3 | (45) | 4.08 | 1.24 | 21.32 | 1.48E−02 | 1.00 | 4.51E−01 |
| | ATP6V1C1 | | | | | | | | | | |
| Spinal Cord - | TVP23C--CDRT4 | 136 | (252) | 11 | (41) | 3.19 | 1.47 | 7.37 | 1.32E−03 | 1.17E−01 | 1.17E−01 |
| Lumbar | | | | | | | | | | | |
| | IRF1-AS1-- | 73 | (252) | 5 | (41) | 2.93 | 1.08 | 9.94 | 2.29E−02 | 1.00 | 7.33E−01 |
| | RAD50 | | | | | | | | | | |
| | AC012405.1-- | 80 | (252) | 6 | (41) | 2.71 | 1.07 | 8.19 | 2.66E−02 | 1.00 | 7.33E−01 |
| | AC073941.1 | | | | | | | | | | |
| Frontal Cortex | AC090517.5-- | 191 | (248) | 26 | (48) | 2.82 | 1.41 | 5.63 | 2.10E−03 | 2.16E−01 | 1.61E−01 |
| | ZNF280D | | | | | | | | | | |
| | TVP23C--CDRT4 | 166 | (248) | 21 | (48) | 2.59 | 1.32 | 5.15 | 3.12E−03 | 3.21E−01 | 1.61E−01 |
| | SH3GL3-- | 30 | (248) | 0 | (48) | 13.54 | 1.61 | Inf | 6.98E−03 | 7.19E−01 | 2.40E−01 |
| | ADAMTSL3 | | | | | | | | | | |
| Temporal Cortex | AEBP2-- | 13 | (34) | 0 | (25) | 32.02 | 3.08 | Inf | 2.74E−04 | 1.12E−02 | 1.12E−02 |
| | AC024901.1 | | | | | | | | | | |
| | TVP23C--CDRT4 | 23 | (34) | 6 | (25) | 6.38 | 1.82 | 25.57 | 1.43E−03 | 5.87E−02 | 2.93E−02 |
| | AC090517.5-- | 23 | (34) | 8 | (25) | 4.32 | 1.30 | 15.64 | 8.98E−03 | 3.68E−01 | 1.23E−01 |
| | ZNF280D | | | | | | | | | | |
| Hippocampus | EFCAB2--KIF26B | 0 | (61) | 3 | (13) | 0.04 | 0 | 0.47 | 4.41E−03 | 2.12E−01 | 2.12E−01 |
| | TVP23C--CDRT4 | 36 | (61) | 4 | (13) | 3.19 | 0.78 | 15.78 | 7.49E−02 | 1.00 | 1.00 |
| | MAILR-- | 18 | (61) | 1 | (13) | 4.94 | 0.64 | 226.24 | 1.63E−01 | 1.00 | 1.00 |
| | ATP6V1C1 | | | | | | | | | | |
| Cerebellum | EEF1AKNMT--DNM3 | 50 | (187) | 2 | (29) | 4.90 | 1.16 | 44.06 | 1.91E−02 | 1.00 | 1.00 |
| | AC090517.5-- | 92 | (187) | 9 | (29) | 2.14 | 0.88 | 5.64 | 7.49E−02 | 1.00 | 1.00 |
| | ZNF280D | | | | | | | | | | |
| | OTUB1-- | 0 | (187) | 1 | (29) | 0.05 | 0.00 | 6.05 | 1.34E−01 | 1.00 | 1.00 |
| | AP000721.2 | | | | | | | | | | |

Intra-chromosomal Fusion Type: Overlapping Neighbor

| | | | | | | | | | | | |
|---|---|---|---|---|---|---|---|---|---|---|---|
| Motor Cortex | AC127502.3-- | 52 | (373) | 2 | (37) | 2.83 | 0.69 | 25.00 | 2.02E−01 | 1.00 | 1.00 |
| | AC127502.1 | | | | | | | | | | |
| | KHDRBS2- | 371 | (373) | 36 | (37) | 5.12 | 0.09 | 100.56 | 2.48E−01 | 1.00 | 1.00 |
| | OT1--KHDRBS2 | | | | | | | | | | |
| | Z68871.1-- | 325 | (373) | 30 | (37) | 1.58 | 0.55 | 3.94 | 3.12E−01 | 1.00 | 1.00 |
| | LINC00630 | | | | | | | | | | |
| Frontal Cortex | AC067956.1-- | 71 | (248) | 4 | (48) | 4.40 | 1.52 | 17.48 | 1.97E−03 | 4.34E−02 | 4.34E−02 |
| | AC019211.1 | | | | | | | | | | |
| | AC127502.3-- | 39 | (248) | 2 | (48) | 4.28 | 1.04 | 37.89 | 3.81E−02 | 8.38E−01 | 4.19E−01 |
| | AC127502.1 | | | | | | | | | | |
| | LINC02263-- | 2 | (248) | 1 | (48) | 0.38 | 0.02 | 23.02 | 4.13E−01 | 1.00 | 1.00 |
| | LINC01378 | | | | | | | | | | |

The individual burden tests of gene fusion events were only performed on fusions within tissues demonstrating significant differences in the number of gene fusions of each intra-chromosomal subtype carried by ALS and control samples from each tissue type. Bonferroni and FDR corrections were based on the total number of fusions observed within the respective tissues.
Abbreviations: ALS, amyotrophic lateral sclerosis; CI, confidence interval; FDR, F discovery rate; n, total number of samples; OR, odds ratio.

### Example 5

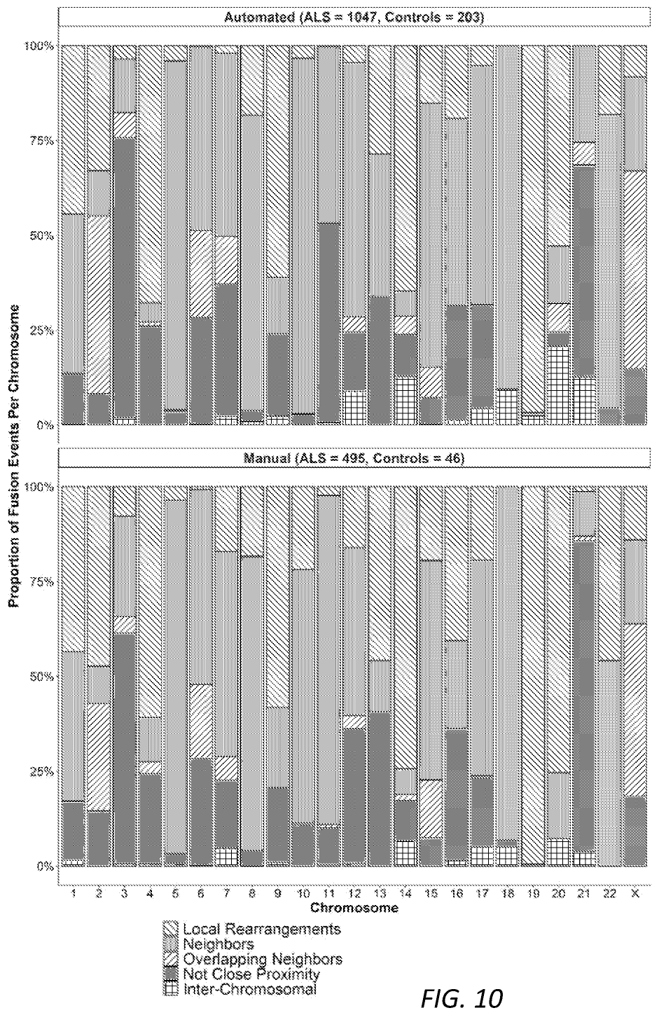

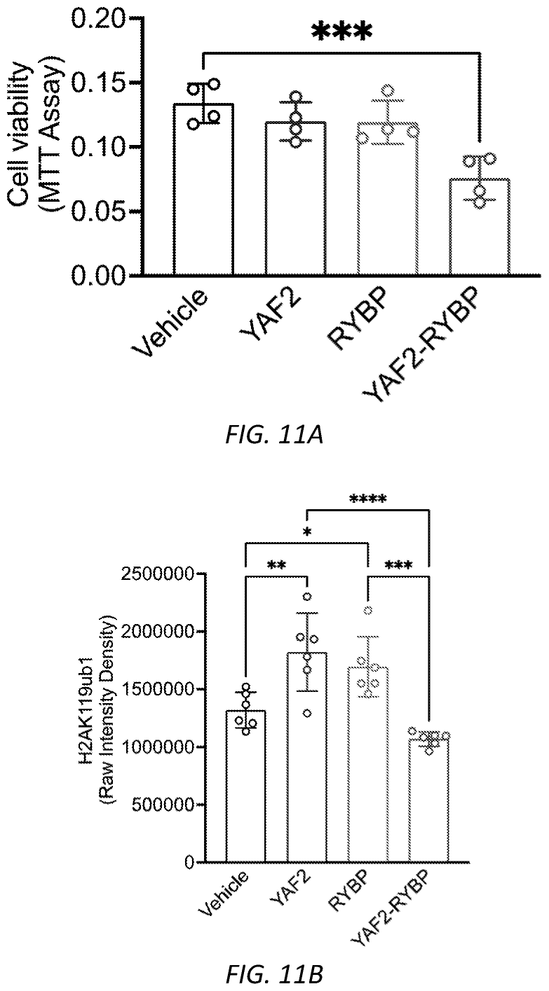

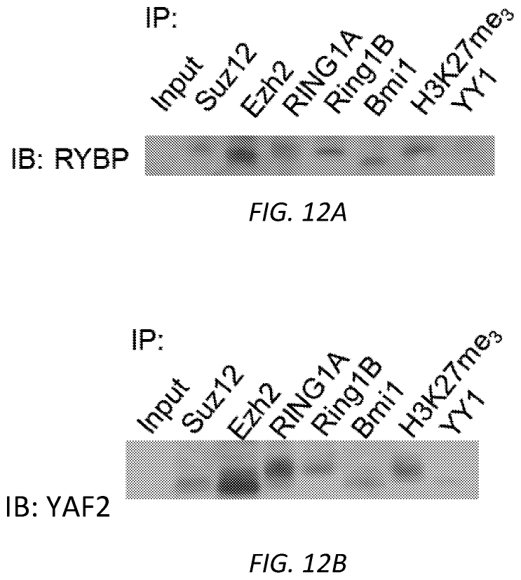

One of the rare inter-chromosomal gene fusion pairs with the highest individual burden in ALS versus control samples was an inter-chromosomal fusion of YAF2-RYBP (Table 2). YAF2 and RYBP regulate chromatin remodeling to form heterochromatin and thereby decrease transcription. These proteins interact with the Polycomb Repressor Complex 1 (PCR1) and YY1 and increase histone mono-ubiquitination. A plasmid was designed to express the YAF2-RYBP fusion and transfected into SH-SY5Y cells. The cells expressed the fusion protein, which significantly decreased cell viability (FIG. 10A) reduced H2AK119ub1 levels (FIG. 10B). In addition, as shown in FIGS. 11A-B. YAF2 and RYBP interact with polycomb complex proteins Ezh2, RING1A and H3K27me₃ in SH-SY5Y cells. These findings suggest that the fusion of YAF2-RYBP interferes with the normal function of the two proteins which is to increase histone H2A mono-ubiquitylation. Furthermore, the presence of the fusion YAF2-RYBP decreased cell viability suggesting a toxic role for the fusion gene. Additional ongoing studies are assessing the effects of the fusion gene on gene expression profiles (RNA-Seq) and chromatin accessibility (ATAC-Seq) to further determine the functional consequences of the fusion gene.

A sequence of an exemplary YAF2-RYBP fusion is the following, however, other fusions of these two genes are possible:

(SEQ ID NO: 1)
ATGGGAGACA AGAAGAGCCC CACCAGGCCA AAAAGACAAG
CGAAACCTGC CGCAGACGAA GGGTTTTGGG ATTGTAGCGT
CTGCACCTTC AGAAACAGTG CTGAAGCCTT TAAATGCAGC
ATCTGCGATG TGAGGAAAGG CACCTCCACC AGAAAACCTC
GGATCAATTC TCAGCTGGTG GCACAACAAG TGGCACAACA
GTATGCCACC CCACCACCCC CTAAAAAGGA GAAGAAGGAG
AAAGTTGAAA AGCAGGACAA AGAGAAACCT GAGAAAGACA
AGGAAATTAG TCCTAGTGTT ACCAAGAAAA ATACCAACAA
GAAAACCAAA CCAAAGTCTG ACATTCTGAA AGATCCTCCT
AGTGAAGCAA ACAGCATACA GTCTGCAAAT GCTACAACAA
AGACCAGCGA AACAAATCAC ACCTCAAGGC CCCGGCTGAA
AAACGTGGAC AGGAGCACTG CACAGCAGTT GGCAGTAACT
GTGGGCAACG TCACCGTCAT TATCACAGAC TTTAAGGAAA
AGACTCGCTC CTCATCGACA TCCTCATCCA CAGTGACCTC
CAGTGCAGGG TCAGAACAGC AGAACCAGAG CAGCTCGGGG
TCAGAGAGCA CAGACAAGGG CTCCTCCCGT TCCTCCACGC
CAAAGGGCGA CATGTCAGCA GTCAATGATG AATCTTTC

### REFERENCES

1. Brown, R. H., & Al-Chalabi, A. (2017). Amyotrophic Lateral Sclerosis. N Engl J Med, 377 (2), 162-172.
2. Ciervo, Y., et al. (2017). Advances, challenges and future directions for stem cell therapy in amyotrophic lateral sclerosis. Mol Neurodegener, 12 (1), 85.
3. Zou, Z. Y., et al. (2017). Genetic epidemiology of amyotrophic lateral sclerosis: a systematic review and meta-analysis. J Neurol Neurosurg Psychiatry, 88 (7), 540-549.
4. Mejzini, R., et al. (2019). ALS Genetics, Mechanisms, and Therapeutics: Where Are We Now? Front Neurosci, 13, 1310.
5. Kim, G., et al. (2020). ALS Genetics: Gains, Losses, and Implications for Future Therapies. Neuron, 108 (5), 822-842.
6. Al Khleifat, A., et al. (2022). Structural variation analysis of 6,500 whole genome sequences in amyotrophic lateral sclerosis. NPJ Genom Med, 7 (1), 8.
7. Sun, Y., et al. (2020). The role of DNA damage response in amyotrophic lateral sclerosis. Essays Biochem, 64 (5), 847-861.
8. Chaudhary, R., Agarwal, V., Rehman, M., Kaushik, A. S., & Mishra, V. (2022). Genetic architecture of motor neuron diseases. J Neurol Sci, 434, 120099.
9. Kok, J. R., et al. (2021). DNA damage as a mechanism of neurodegeneration in ALS and a contributor to astrocyte toxicity. Cell Mol Life Sci, 78 (15), 5707-5729.
10. Wang, H., et al. (2021). DNA Damage and Repair Deficiency in ALS/FTD-Associated Neurodegeneration: From Molecular Mechanisms to Therapeutic Implication. Front Mol Neurosci, 14, 784361.
11. Madabhushi, R., et al. (2014). DNA damage and its links to neurodegeneration. Neuron, 83 (2), 266-282.
12. Lu, T., et al. (2004). Gene regulation and DNA damage in the ageing human brain. Nature, 429 (6994), 883-891.
13. Oliver, G. R., et al. (2020). Computational Detection of Known Pathogenic Gene Fusions in a Normal Tissue Database and Implications for Genetic Disease Research. Front Genet, 11, 173.
14. Mitra, J., et al. (2019). Motor neuron disease-associated loss of nuclear TDP-43 is linked to DNA double-strand break repair defects. Proc Natl Acad Sci US A, 116 (10), 4696-4705.
15. Frenkel-Morgenstern, M., et al. (2012). Chimeras taking shape: potential functions of proteins encoded by chimeric RNA transcripts. Genome Res, 22 (7), 1231-1242.
16. Latysheva, N. S., & Babu, M. M. (2016). Discovering and understanding oncogenic gene fusions through data intensive computational approaches. Nucleic Acids Res, 44 (10), 4487-4503.
17. Taniue, K., & Akimitsu, N. (2021). Fusion Genes and RNAs in Cancer Development. Noncoding RNA, 7 (1). doi: 10.3390/ncrna7010010
18. Zhang, H., et al. (2020). Complex roles of cAMP-PKA-CREB signaling in cancer. Exp Hematol Oncol, 9 (1), 32.
19. Honeyman, J. N., et al (2014). Detection of a recurrent DNAJB1-PRKACA chimeric transcript in fibrolamellar hepatocellular carcinoma. Science, 343 (6174), 1010-1014.
20. You, G., et al. (2021). Fusion Genes Altered in Adult Malignant Gliomas. Front Neurol, 12, 715206.
21. Dai, X., et al. (2018). Fusion genes: A promising tool combating against cancer. Biochim Biophys Acta Rev Cancer, 1869 (2), 149-160.
22. Ferguson, S. D., et al. (2018). Targetable Gene Fusions Associate With the IDH Wild-Type Astrocytic Lineage in Adult Gliomas. J Neuropathol Exp Neurol, 77 (6), 437-442.
23. Haas, B. J., et al. (2019). Accuracy assessment of fusion transcript detection via read-mapping and de novo fusion transcript assembly-based methods. Genome Biol, 20 (1), 213.
24. Dobin, A., et al (2013). STAR: ultrafast universal RNA-seq aligner. Bioinformatics, 29 (1), 15-21.
25. Dobin, A., & Gingeras, T. R. (2015). Mapping RNA-seq Reads with STAR. Curr Protoc Bioinformatics, 51, 11 14 11-11 14 19. doi: 10.1002/0471250953.bi1114s51
26. Dobin, A., & Gingeras, T. R. (2016). Optimizing RNA-Seq Mapping with STAR. Methods Mol Biol, 1415, 245-262.
27. Wickman H. (2009). ggplot2: elegant graphics for data analysis. Springer-Verlag New York. ISBN 978-3-319-24277-4. Link.springer.com/book/10.1007/978-0-387-98141-3.
28. Yu, Y., Ouyang, Y., & Yao, W. (2018). shinyCircos: an R/Shiny application for interactive creation of Circos plot. Bioinformatics, 34 (7), 1229-1231.
29. Pedegrosa F., et al (2011). Scikit-learn: Machine Learning in Python. J Machine Learning Res, 12, 2825-2830.
30. Hunter J. D. (2007). Matplotlib: a 2D graphics environment. In Computing in Science & Engineering, vol. 9, 3, 90-95.
31. Edwards, P. A. (2010). Fusion genes and chromosome translocations in the common epithelial cancers. J Pathol, 220 (2), 244-254.
32. Wang, Z., et al. (2017). Significance of the TMPRSS2: ERG gene fusion in prostate cancer. Mol Med Rep, 16 (4), 5450-5458.
33. Mehani, B., et al. (2020). Fusion transcripts in normal human cortex increase with age and show distinct genomic features for single cells and tissues. Sci Rep, 10 (1), 1368.
34. Heyer, E. E., et al. (2019). Diagnosis of fusion genes using targeted RNA sequencing. Nat Commun, 10 (1), 1388.
35. Hehir-Kwa, J. Y., et al. (2022). Improved Gene Fusion Detection in Childhood Cancer Diagnostics Using RNA Sequencing. JCO Precis Oncol, 6, e2000504.
36. Latysheva, N. S., & Babu, M. M. (2019). Molecular Signatures of Fusion Proteins in Cancer. ACS Pharmacol Transl Sci, 2 (2), 122-133.
37. Farg, M. A., et al. (2017). The DNA damage response (DDR) is induced by the C9orf72 repeat expansion in amyotrophic lateral sclerosis. Hum Mol Genet, 26 (15), 2882-2896.
38. Zhang, W. H., et al. (2020). The Significance of the CLDN18-ARHGAP Fusion Gene in Gastric Cancer: A Systematic Review and Meta-Analysis. Front Oncol, 10, 1214.
39. Karki, A., et al. (2019). MDM4 expression in fibrolamellar hepatocellular carcinoma. Oncol Rep, 42 (4), 1487-1496.
40. Shi, Y., et al. (2021). Aberrant splicing in neuroblastoma generates RNA-fusion transcripts and provides vulnerability to spliceosome inhibitors. Nucleic Acids Res, 49 (5), 2509-2521.
41. Oliver, G. R., et al. (2019). A tailored approach to fusion transcript identification increases diagnosis of rare inherited disease. PLOS One, 14 (10), e0223337.
42. Oliver, G. R., et al. (2019). RNA-Seq detects a SAMD12-EXT1 fusion transcript and leads to the discovery of an EXT1 deletion in a child with multiple osteochondromas. Mol Genet Genomic Med, 7 (3), e00560.
43. Cousin, M. A., et al. (2018). Utility of DNA, RNA, Protein, and Functional Approaches to Solve Cryptic Immunodeficiencies. J Clin Immunol, 38 (3), 307-319.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method comprising: providing a sample, preferably a sample comprising genomic DNA (gDNA) or RNA, optionally non-coding mRNA and/or mRNA, from a human subject, and detecting presence of a fusion described in any of Tables 2-7 in the gDNA or mRNA.

2. The method of claim 1, wherein the fusion is a fusion of YAF2 and RYBP genes.

3. The method of claim 1, wherein the subject is suspected of having or at risk of having amyotrophic lateral sclerosis (ALS).

4. A method of diagnosing amyotrophic lateral sclerosis (ALS) or risk of developing ALS in a subject, the method comprising detecting presence of a fusion described in any of Tables 2-7.

5. The method of claim 4, wherein the fusion is a fusion of YAF2 and RYBP genes.

6. The method of claim 1, wherein the sample comprises serum or cerebrospinal fluid, or comprises gDNA or RNA isolated from whole blood, plasma, serum or cerebrospinal fluid.

7. The method of claim 5, wherein the method further comprises isolating exosomes comprising proteins or nucleic acids from the sample, and detecting the presence of a fusion in the exosomes.

8. The method of claim 1, wherein detecting presence of a fusion comprises using RNA-seq and/or long-read sequencing to detect RNA transcripts of a fusion.

9. The method of claim 1, wherein detecting presence of a fusion comprises contact the sample with a plurality of oligonucleotides that bind to a fusion sequence.

10. The method of claim 9, wherein the fusion comprises a fusion of two or more genes, and the plurality comprises oligonucleotides that bind to each of the two or more genes.

11. The method of claim 10, wherein the fusion is a fusion of YAF2 and RYBP genes, and the plurality comprises oligonucleotides that bind to YAF2 and oligonucleotides that bind to RYBP.

12. The method of claim 9, wherein each of the plurality of oligonucleotides comprise a primer for sequencing, and the method comprises determining a sequence of a portion of the gDNA and/or RNA.

13. The method of claim 9, wherein each of the plurality of oligonucleotides is suitable for priming amplification, and the method further comprises amplifying a portion of the gDNA and/or RNA using oligonucleotides bound to the gDNA and/or RNA as primers.


SEQUENCE LISTING (1 sequence)

SEQ ID NO: 1 - DNA, 678 bp, synthetic construct
  source 1..678: mol_type = other DNA
atgggagacaagaagagccccaccaggccaaaaagacaagcgaaacctgccgcagacgaagggttttgggattgtagcgtctgcaccttcagaaacagtgctgaagcctttaaatgcagcatctgcgatgtgaggaaaggcacctccaccagaaaacctcggatcaattctcagctggtggcacaacaagtggcacaacagtatgccaccccaccaccccctaaaaaggagaagaaggagaaagttgaaaagcaggacaaagagaaacctgagaaagacaaggaaattagtcctagtgttaccaagaaaaataccaacaagaaaaccaaaccaaagtctgacattctgaaagatcctcctagtgaagcaaacagcatacagtctgcaaatgctacaacaaagaccagcgaaacaaatcacacctcaaggccccggctgaaaaacgtggacaggagcactgcacagcagttggcagtaactgtgggcaacgtcaccgtcattatcacagactttaaggaaaagactcgctcctcatcgacatcctcatccacagtgacctccagtgcagggtcagaacagcagaaccagagcagctcggggtcagagagcacagacaagggctcctcccgttcctccacgccaaagggcgacatgtcagcagtcaatgatgaatctttc